(19) United States
(12) Patent Application Publication
(10) Pub. No.: US 2026/0083736 A1
(43) Pub. Date: Mar. 26, 2026

(54) GHRELIN O-ACYL TRANSFERASE (GOAT) INHIBITORS FOR USE IN THE TREATMENT OF SUBSTANCE USE DISORDER IN PATIENTS WITH HIGH IMPULSIVITY

(71) Applicant: Boehringer Ingelheim International GmbH, Ingelheim am Rhein (DE)

(72) Inventors: Stefan JUST, Biberach an der Riss (DE); Hugo CRUCES SOLIS, Berlin (DE); Johann Faustus DU HOFFMANN, Biberach an der Riss (DE); Jennifer Buenzle DWYER, Branford, CT (US); Janet Rachel NICHOLSON, Oberhoefen (DE); Katrin Hildegund PRELLER, Oberweningen (CH)

(21) Appl. No.: 19/335,420

(22) Filed: Sep. 22, 2025

(30) Foreign Application Priority Data
Sep. 24, 2024 (EP) 24202338.0

(51) Int. Cl. A61K 31/506; A61K 31/437; A61K 31/444; A61K 31/497; A61K 31/5377; A61K 31/551

(52) U.S. Cl. CPC A61K 31/506; A61K 31/437; A61K 31/444; A61K 31/497; A61K 31/5377; A61K 31/551

(57) ABSTRACT

The present invention relates to compounds of general formula I,

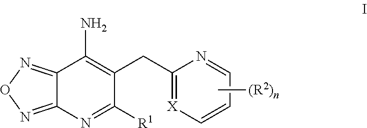

wherein the groups R¹, R² and n are defined herein, which are ghrelin O-acyl transferase (GOAT) inhibitors. The invention also relates to the use of compounds of formula (I) for treatment of substance use disorder (SUD,) in particular for use in the treatment of opioid use disorder (OUD) or stimulant use disorder (StimUD), in patients with high impulsivity.

### FIELD OF THE INVENTION

The present invention relates to oxadiazolopyridine derivatives that are inhibitors of the ghrelin O-acyl transferase (GOAT), and to pharmaceutical compositions containing these compounds, for use in the treatment of substance use disorder (SUD) including both opioid use disorder (OUD) and stimulant use disorder (StimUD), in patients with high impulsivity.

### BACKGROUND OF THE INVENTION

Up to 40 million Americans are impacted by substance use disorders (SUDs), with a high associated personal, familial, and societal burden. Opioid use disorder (OUD) is estimated to impact from 3 million to 5.5 million Americans and up to 16 million people worldwide. Stimulant use disorder (StimUD) is estimated to impact 6.5 million Americans, with global estimates of 7.4 million people with amphetamine dependence and 5 million with cocaine dependence.

Impulsivity is a neuroendophenotype that can be defined as a “pre-disposition toward rapid, unplanned reactions to internal or external stimuli without regard to the negative consequences of these reactions to the impulsive individual or to others” (Moeller F G, Barratt E S, Dougherty D M, et al. Psychiatric aspects of impulsivity, Am J Psychiatry; 2001; 158(11); 1783-1793). Impulsivity plays an important role in all phases of the addiction lifecycle: high trait impulsivity is associated with a higher risk of conversion from casual drug use to compulsive drug taking (Jupp B, Dailey J W, Behavioral endophenotypes of drug addiction: etiological insights from neuroimaging studies. Neuropharmacology 2014; 76; 487-497). High impulsivity is also seen during active addiction including risky and unpredictable behavior while intoxicated or while seeking drugs (Schaub A C, Vogel M, Lang U E, et al. Transdiagnostic brain correlates of self-reported trait impulsivity: a dimensional structure-symptom investigation. Neuroimage Clin 2023; 38; 103423; Lahbbairi N, Laniepce A, Segobin S, et al. Determinants of health-related quality of life in recently detoxified patients with severe alcohol use disorder. Health Qual Life Outcomes; 2022; 20; 149). Finally, impulsivity also plays a critical role in the risk of relapse during abstinence (Jupp B, Dailey J W, Behavioral endophenotypes of drug addiction: etiological insights from neuroimaging studies. Neuropharmacology 2014; 76; 487-497). In particular, cue reactivity, defined as the subjective, neural, physiologic, and behavioral associations to drug-associated cues (Drummond D C: What does cue-reactivity have to offer clinical research?Addiction 2000; 95 (Suppl 2); S129-S144), and the impulsive responses to them play a critical role in relapse (Vafaie N, Kober H. Association of drug cues and craving with drug use and relapse: a systematic review and meta-analysis: JAMA Psychiatry 2022; 797:641-650). It is hypothesized that SUD patients with high impulsivity may both be more vulnerable to drug-associated cues, as well as less able to resist the impulses that those cues generate by engagement of top-down neural circuitry (Cunningham K A, Anastasio N C. Serotonin at the nexus of impulsivity and cue reactivity in cocaine addiction. Neuropharmacology 2014; 76:460-478). Thus, patients with high impulsivity are a particularly vulnerable and high-risk subgroup within SUDs more broadly.

Despite increased efforts at treatment and prevention, OUD and StimUD, particularly in the United States, have only grown in incidence and show few signs of improvement. Since the 1990s, OUD incidence and overdose deaths via opioids have reached epidemic proportions. The number of people who died from a drug overdose in 2021 was over 6× that seen in 1999. From 2020 to 2021 alone, drug overdose deaths increased more than 16%. While opioid overdoses are the most common drug-associated cause of mortality, stimulant-associated deaths are not far behind. From 2013 through 2018, the age-adjusted rate of cocaine-associated overdose fatalities nearly tripled- an increase by about 27% per year. The costs of SUDs are not only reflected in individual FIGURES relating to health care resources utilization (HCRU), morbidity, and mortality, but also pay a heavy toll on families and society at large. These broader impacts can be captured in escalating rates of children in the foster-care system, drug-associated arrests and crimes, escalating rates of unhoused people with SUDs, and the costs to local and federal governments to manage these crises.

Ghrelin O-Acyltransferase (GOAT) is a member of the membrane-bound O-acyl transferase (MBOAT) protein family, and the only enzyme in humans capable of promoting an acylation reaction on the peptide hormone ghrelin. By linking a medium-chain fatty acid to the Serine-3 position of the 28-amino acid peptide, GOAT converts unacylated ghrelin (UAG) to acylated ghrelin (AG) which is the natural ligand of the ghrelin receptor GHSR1a (growth hormone secretagogue receptor 1a).

The enzyme ghrelin o-acyl transferase (GOAT) leads to reduced levels of the activated form of ghrelin (acylated ghrelin). Acyl-ghrelin (AG) can cross the blood brain barrier and bind to its receptor in the brain, where it increases the excitability of dopaminergic neurons that modulate the basal ganglia, in key areas that regulate impulsive behavior and cue-related reward responding. While classically studied as a feeding peptide, ghrelin levels and ghrelin signaling have since been implicated in the pathophysiology of addiction (Zallar L J, Farokhnia M, Tunstall B J, et al. The role of the ghrelin system in drug addiction. Int Rev Neurobiol; 2017; 136; 89-119; Engel J A, Nylander I, Jerlhag E. A ghrelin receptor (GHS-R¹a) antagonist attenuates the rewarding properties of morphine and increases opioid peptide levels in reward areas in mice. Eur Neuropsychopharmacol; 2015; 25; 2364-2371) and the expression of impulsive behaviors (Ralevski E, Shanabrough M, Newcomb J, et al. Ghrelin is related to personality differences in reward sensitivity and impulsivity. Alcohol Alcohol 2018; 531:52-56; Anderberg R H, Hansson C, Fenander M, et al. The stomach-derived hormone ghrelin increases impulsive behavior. Neuropsychopharmacology; 2016; 41; 1199-1209)).

WO 2018/024653 discloses ghrelin O-acyl transferase (GOAT) inhibitors of formula (I), their preparation and certain details regarding their biological properties.

### Object of the Present Invention

The object of the present invention is to provide compounds, hereinafter described as compounds of formula I, in particular oxadiazolopyridine derivatives, which are ghrelin O-acyl transferase (GOAT) inhibitors for use in the treatment of substance use disorder (SUD) in patients with high impulsivity, particularly for use in the treatment of opioid use disorder (OUD) in patients with high impulsivity and for use in the treatment of stimulant use disorder (StimUD) in patients with high impulsivity.

A further object of the present invention is to provide a pharmaceutical composition comprising at least one compound of formula (I) for use in the treatment of substance use disorder (SUD) in patients with high impulsivity, particularly for use in the treatment of opioid use disorder (OUD) in patients with high impulsivity or for use in the treatment of stimulant use disorder (StimUD) in patients with high impulsivity.

A further object of the present invention is to provide a combination of at least one compound of formula (I) with one or more additional therapeutic agents for use in the treatment of substance use disorder (SUD) in patients with high impulsivity, particularly for use in the treatment of opioid use disorder (OUD) in patients with high impulsivity or for use in the treatment of stimulant use disorder (StimUD) in patients with high impulsivity.

A further object of the present invention is to provide the use of a compound of formula I or a pharmaceutical composition comprising a compound of formula I for the treatment of substance use disorder (SUD) in patients with high impulsivity, particularly for use in the treatment of opioid use disorder (OUD) in patients with high impulsivity or for use in the treatment of stimulant use disorder (StimUD) in patients with high impulsivity, wherein preferably, the patients are humans.

A further object of the present invention is to provide a method for treating substance use disorder (SUD) in patients with high impulsivity, particularly for use in the treatment of opioid use disorder (OUD) in patients with high impulsivity or for use in the treatment of stimulant use disorder (StimUD) in patients with high impulsivity, wherein the patients are preferably mammals, and the method includes the step of administering to a patient, preferably a human, in need of such treatment a therapeutically effective amount of a compound or a pharmaceutical composition comprising a compound fo formula I.

A further object of the present invention is to provide the use of a compound of formula I in the manufacture of a medicament for treating substance use disorder (SUD) in patients with high impulsivity, particularly for use in the treatment of opioid use disorder (OUD) in patients with high impulsivity or for use in the treatment of stimulant use disorder (StimUD) in patients with high impulsivity.

Further objects of the present invention will become apparent to the one skilled in the art from the entirety of the disclosure and the examples.

### SUMMARY OF THE INVENTION

In a first aspect, the invention relates to a compound of formula

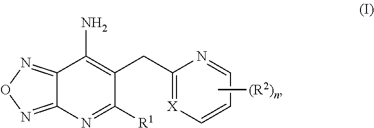

wherein
X is CH or N;
R¹ is selected from the group R¹-G1 consisting of CH₃, —CH₂OH and Cl;
R² is independently of each other selected from the group R²-G1 consisting of H, F, Cl, Br, I, CN, C₁₋₆-alkyl, C₃₋₇-cycloalkyl, OH, —O—(C₁₋₆-alkyl), —O—(C₃₋₇-cycloalkyl), —O—(C₁₋₃-alkyl)-(C₃₋₇-cycloalkyl), —O-heterocyclyl, —O—(C₁₋₃-alkyl)-heterocyclyl, —O-aryl, —O-heteroaryl, —S—(C₁₋₃-alkyl), —SO—(C₁₋₃-alkyl), —SO₂-(C₁₋₃-alkyl), —NH₂, —NH—(C₁₋₆-alkyl), —NH—(C₃₋₆-cycloalkyl), —NH—(C₁₋₃-alkyl)-heterocyclyl, —NH—(C₁₋₆-alkyl)-C(═O)—NH₂, —C(═O)—NH₂, —C(═O)—NH—(C₁₋₃-alkyl), —C(═O)—N(C₁₋₃-alkyl)₂, —C(═O)OH, —C(═O)—O—(C₁₋₄-alkyl), —C(═O)-(C₁₋₄-alkyl), —C₁₋₃-alkyl-C(═O)—O—(C₁₋₄-alkyl), heterocyclyl, heteroaryl and 5,6-dihydro-4H-pyrrolo[1,2-b]pyrazol-3-yl,
   wherein each alkyl or cycloalkyl group is optionally independently substituted with one or more substituents selected from the group consisting of F, CN and OH, and
   wherein each heterocyclyl group is selected from a mono- or spirocyclic 4-7-membered cycloalkyl group, in which 1, 2 or 3 CH₂-groups are independently of each other replaced by O, S, NH or C═O, and
   wherein each heterocyclyl group is optionally substituted with 1 or 2 substituents independently of each other selected from F, OH and C₁₋₃-alkyl,
   wherein each aryl group is selected from a group consisting of phenyl and naphthyl, and
   wherein each heteroaryl group is selected from a 5-membered aromatic cycle containing 1 or 2 heteroatoms independently selected from N, O and S or from a 6-membered aromatic cycle containing 1 or 2 N, and
   wherein each aryl or heteroaryl group is optionally substituted with 1 or 2 substituents independently selected from a group consisting of F, CN and C₁₋₃-alkyl, which is optionally substituted with one or more F;
or, if two groups R² are attached to adjacent C atoms of the pyridine or pyrimidine group, they may be linked with each other and together form a —O—CH₂—O—, —O—CH₂—CH₂—O— or —O—CH₂—CH₂—CH₂—O— bridge, in which 1 or 2 H atoms may be replaced with F or C₁₋₃-alkyl; and
n is 1, 2 or 3;
wherein each of the above-mentioned alkyl groups may be substituted with one or more F;
the isoforms, tautomers, stereoisomers, metabolites, prodrugs, solvates, hydrates, and the salts thereof, particularly the physiologically acceptable salts thereof with inorganic or organic acids or bases, or the combinations thereof,
for use in the treatment of substance use disorder (SUD) in patients with high impulsivity, particularly for use in the treatment of opioid use disorder (OUD) or stimulant use disorder (StimUD), both in patients with high impulsivity.

The treatment with a compound of formula (I) may be adjunctive to the standard of care, such as the treatment of an OUD patient with buprenorphine, methadone, L-methadone or naltrexone, particularly with buprenorphine.

The extension -Gn used within the definitions is meant to identify genus n of the respective substituent. For example, R-G1 defines genus 1 of the substituent R.

The expression “optionally substituted with 1 or more F atoms” means that none or one up to successively all H atoms bound to carbon atoms of the respective group or submoiety may be replaced by F atoms, preferably 1 to 5 H atoms or, more preferred, 1 to 3 H atoms may be replaced by F atoms.

In a further aspect this invention relates to a pharmaceutical composition, comprising one or more compounds of general formula I or one or more pharmaceutically acceptable salts thereof according to the invention, optionally together with one or more inert carriers and/or diluents, for use in the treatment of substance use disorder (SUD) in patients with high impulsivity, particularly for use in the treatment of opioid use disorder (OUD) or stimulant use disorder (StimUD), both in patients with high impulsivity.

In a further aspect this invention relates to a method for treating substance use disorder (SUD), particularly treating opioid use disorder (OUD) or stimulant use disorder (StimUD), in a patient with high impulsivity in need thereof characterized in that a compound of general formula I or a pharmaceutically acceptable salt thereof is administered to the patient.

According to another aspect of the invention, there is provided a method for treating substance use disorder (SUD), particularly for treating opioid use disorder (OUD) or stimulant use disorder (StimUD), in a patient with high impulsivity in need thereof characterized in that a therapeutically effective amount of a compound of general formula I or a pharmaceutically acceptable salt thereof is administered to the patient.

According to another aspect of the invention, there is provided the use of a compound of the general formula I or a pharmaceutically acceptable salt thereof for the manufacture of a medicament for a therapeutic method as described hereinbefore and hereinafter.

According to another aspect of the invention, there is provided a compound of the general formula I or a pharmaceutically acceptable salt thereof for use in a therapeutic method as described hereinbefore and hereinafter.

In a further aspect this invention relates to a method for treating substance use disorder (SUD), particularly for treating opioid use disorder (OUD) or stimulant use disorder (StimUD), in a patient with high impulsivity that includes the step of administering to the patient in need of such treatment a therapeutically effective amount of a compound of the general formula I or a pharmaceutically acceptable salt thereof in combination with a therapeutically effective amount of one or more additional therapeutic agents.

In a further aspect this invention relates to the use of a compound of the general formula I or a pharmaceutically acceptable salt thereof in combination with one or more additional therapeutic agents for the treatment of substance use disorder (SUD), particularly for treating opioid use disorder (OUD) or stimulant use disorder (StimUD), in patients with high impulsivity.

In a further aspect this invention relates to a pharmaceutical composition which comprises a compound according to general formula I or a pharmaceutically acceptable salt thereof and one or more additional therapeutic agents, optionally together with one or more inert carriers and/or diluents, for use in the treatment of substance use disorder (SUD), particularly for use in the treatment of opioid use disorder (OUD) or stimulant use disorder (StimUD), in patients with high impulsivity.

In addition, the present invention relates to a compound of formula I or a pharmaceutical composition comprising a compound of formula I for use in
Improving impulse control,
Normalizing impulsivity, particularly in substance use disorder patients with high impulsivity,
Improving the results of impulsivity tasks or scales such as the Delay Discounting Task (DDT), the 4-Choice Serial Reaction Time Test (4-CSRTT), the Barratt Impulsiveness Scale, version 11 (BIS-11), the Short Urgency, Premeditation, Perseverance, Sensation Seeking, Positive Urgency Impulsive Behavior Scale (S—UPPS-P) and/or the Opioid Craving Visual Analog Scale (OC-VAS),
Reducing or normalizing cue reactivity, particularly in substance use disorder patients with high impulsivity,
Reducing or normalizing addictive behaviors, particularly in substance use disorder patients with high impulsivity,
Reducing relapse after abstinence,
Reducing drug intake,
Improving quality of life,
Improving adherence and compliance with medications,
Improving adherence and compliance to psychotherapy,
Decreasing overall mortality and overdose mortality,
Reducing criminal behavior to satisfy drug consumption, and/or
Reducing craving, reducing drug use or abstaining from drug use or changing drug use pattern, wherein drug includes opioids and stimulants,
   in SUD, OUD or StimUD patients, particularly in SUD, OUD or StimUD patients with high impulsivity.

The above use of a compound of formula (I) may be adjunctive to the standard of care of the treated patients. For example, OUD patients to be treated with a compound of formula (I) to achieve one of the above goals may be may also be taking buprenorphine, methadone, L-methadone or naltrexone, particularly buprenorphine.

In a preferred embodiment, the present invention relates to a compound of formula I or a pharmaceutical composition comprising a compound of formula I for use in
Normalizing impulsivity, particularly in substance use disorder patients with high impulsivity,
Reducing or normalizing cue reactivity, particularly in substance use disorder patients with high impulsivity,
Reducing or normalizing addictive behaviors, particularly in substance use disorder patients with high impulsivity,
Reducing relapse after abstinence,
Reducing drug intake,
Improving quality of life,
Improving adherence and compliance with medications
Improving adherence and compliance to psychotherapy,
Decreasing overall mortality and overdose mortality,
Reducing criminal behavior to satisfy drug consumption, and/or
Reducing craving, reducing drug use or abstaining from drug use or changing drug use pattern, wherein drug includes opioids and stimulants,
   in SUD, OUD or StimUD patients, particularly in SUD, OUD or StimUD patients with high impulsivity.

In a more preferred embodiment, the present invention relates to a compound of formula I or a pharmaceutical composition comprising a compound of formula I for use in
Reducing or normalizing addictive behaviors, particularly in substance use disorder patients with high impulsivity,
Reducing relapse after abstinence, and/or
Reducing drug intake,
   in SUD, OUD or StimUD patients, particularly in SUD, OUD or StimUD patients with high impulsivity.

Another aspect of the present invention relates to a compound of formula I or a pharmaceutical composition comprising a compound of formula I for use in the treatment of alcohol use disorder in patients with high impulsivity.

Furthermore, the present invention relates to a compound of formula I or a pharmaceutical composition comprising a compound of formula I for use in the treatment of behavioral addiction such as gambling disorder in patients with high impulsivity.

Other aspects of the invention become apparent to the one skilled in the art from the specification and the experimental part as described hereinbefore and hereinafter.

### DETAILED DESCRIPTION

Unless otherwise stated, the groups, residues, and substituents, particularly X, R¹, R² and n are defined as above and hereinafter. If residues, substituents, or groups occur several times in a compound, they may have the same or different meanings. Some preferred meanings of individual groups and substituents of the compounds according to the invention will be given hereinafter. Any and each of these definitions may be combined with each other.

### X:

X is preferably CH or N.
According to one embodiment, X is CH.
According to another embodiment, X is N.

### R¹;

R¹-G1:
The group R¹ is preferably selected from the group R¹-G1 as defined hereinbefore.

### R¹-G2:

In one embodiment the group R¹ is selected from the group R¹-G2 consisting of CH₃ and C₁.

### R¹-G3:

In another embodiment the group R¹ is selected from the group R¹-G3 consisting of CH₃ and —CH₂OH.

### R¹-G4:

In another embodiment the group R¹ is selected from the group R¹-G4 consisting of —CH₂OH and C₁.

### R¹-G5:

In another embodiment the group R¹ is selected from the group R¹-G5 consisting of CH₃.

### R¹-G6:

In another embodiment the group R¹ is selected from the group R¹-G6 consisting of —CH₂OH.

### R¹-G7:

In another embodiment the group R¹ is selected from the group R¹-G7 consisting of C₁.

### R²:

### R²-G1:

The group R² is preferably selected from the group R²-G1 as defined hereinbefore.

### R²-G2:

In another embodiment the group R² is independently of each other selected from the group R²-G2 consisting of H, F, Cl, Br, CN, C₁₋₆-alkyl, C₃₋₇-cycloalkyl, OH, —O—(C₁₋₆-alkyl), —O—(C₁₋₃-alkyl)-(C₃₋₇-cycloalkyl), —O-heterocyclyl, —O—(C₁₋₃-alkyl)-heterocyclyl, —O-aryl, —O-heteroaryl, —S—(C₁₋₃-alkyl), —SO₂-(C₁₋₃-alkyl), —NH₂, —NH—(C₁₋₆-alkyl), —NH—(C₃₋₆-cycloalkyl), —NH—(C₁₋₃-alkyl)-heterocyclyl, —NH—(C₁₋₆-alkyl)-C(═O)—NH₂, —C(═O)—NH₂, —C(═O)—NH—(C₁₋₃-alkyl), —C(═O)-(C₁₋₄-alkyl), —C₁₋₃-alkyl-C(═O)—O—(C₁₋₄-alkyl), heterocyclyl, heteroaryl and 5,6-dihydro-4H-pyrrolo[1,2-b]pyrazol-3-yl,
   wherein each alkyl or cycloalkyl group is optionally independently substituted with one or more substituents selected from the group consisting of F, CN and OH, and
   wherein each heterocyclyl group is selected from a mono- or spirocyclic 4-7-membered cycloalkyl group, in which 1, 2 or 3 CH₂-groups are independently of each other replaced by O, S, NH or C═O, and
   wherein each heterocyclyl group is optionally substituted with 1 or 2 substituents independently of each other selected from F, OH and C₁₋₃-alkyl,
   wherein each aryl group is selected from a group consisting of phenyl and naphthyl, and
   wherein each heteroaryl group is selected from a 5-membered aromatic cycle containing 1 or 2 heteroatoms independently selected from N, O and S or from a 6-membered aromatic cycle containing 1 or 2 N, and
   wherein each aryl or heteroaryl group is optionally substituted with 1 or 2 substituents independently selected from a group consisting of F and C₁₋₃-alkyl, which is optionally substituted with one or more F;
      or, if two groups R² are attached to adjacent C atoms of the pyridine or pyrimidine group, they may be linked with each other and together form a —O—CH₂—O—, —O—CH₂—CH₂—O— or —O—CH₂—CH₂—CH₂—O— bridge.

### R²-G3:

In another embodiment the group R² is independently of each other selected from the group R²-G3 consisting of F, Cl, Br, CN, C₁₋₃-alkyl, C₃₋₆-cycloalkyl, —O—(C₁₋₄-alkyl), —O—CH₂-cyclopropyl, —O—CH₂-heterocyclyl, —O-phenyl, —O-heteroaryl,-S—CH₃, —NH₂, —NH—(C₁₋₄-alkyl), —NH—(C₃₋₅-cycloalkyl), —NH—(CH₂-heterocyclyl), —NH—(C₁₋₄-alkyl)-C(═O)—NH₂, —C(═O)—NH—(C₁₋₃-alkyl), —C(═O)-(C₁₋₄-alkyl), heterocyclyl, heteroaryl and 5,6-dihydro-4H-pyrrolo[1,2-b]pyrazol-3-yl,
   wherein each alkyl or cycloalkyl group is optionally independently substituted with one to three F atoms or with one CN or one OH, and
   wherein each heterocyclyl group is selected from a group consisting of oxetanyl, tetrahydrofuranyl, azetidinyl, pyrrolidinyl, morpholinyl and 1,4-diazepan-5-one, and
   wherein each heterocyclyl group is optionally substituted with 1 or 2 substituents independently of each other selected from F, OH and CH₃,
   wherein each heteroaryl group is selected from a group consisting of furanyl, isoxazolyl, thiazolyl and pyrazolyl, and
   wherein each heteroaryl group is optionally substituted with 1 or 2 substituents independently selected from a group consisting of F, CH₃ and CF₃.

### R²-G4:

In another embodiment the group R² is independently of each other selected from the group R²-G4 consisting of F, Cl, Br, CN, CH₃, C₃₋₅-cycloalkyl, —O—(C₁₋₄-alkyl), —O—CH₂-heterocyclyl, —O-phenyl,-S—CH₃, —NH₂, —NH—(C₁₋₄-alkyl), —NH—(C₃₋₅-cycloalkyl), —NH—(CH₂-heterocyclyl), —NH—(C₁₋₄-alkyl)-C(═O)—NH₂, heterocyclyl, heteroaryl and 5,6-dihydro-4H-pyrrolo[1,2-b]pyrazol-3-yl,
   wherein each alkyl or cycloalkyl group is optionally independently substituted with one to three F atoms or with one CN or one OH, and
   wherein each heterocyclyl group is selected from a group consisting of oxetanyl, azetidinyl, pyrrolidinyl, morpholinyl and 1,4-diazepan-5-one, and
   wherein each heterocyclyl group is optionally substituted with 1 or 2 substituents independently selected from F, OH and CH₃, and
   wherein each heteroaryl group is selected from a group consisting of furanyl and thiazolyl.

### R²-G5:

In another embodiment the group R² is independently selected from the group R²-G5 consisting of:

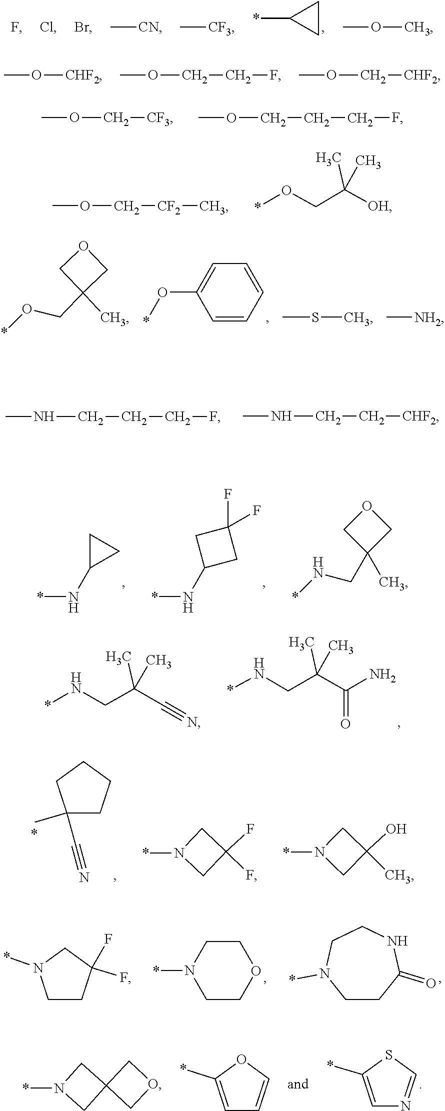

In another embodiment the group R² is independently selected from the group R²-G6 consisting of H, F, Cl, Br, CN, —CF₃, —CHF₂, —CH₂F, —O-CF₃, —O—CHF₂, —O—CH₂F, —O—CH₃, —NH₂, —CO—NH₂, —CO₂H.

n
The index n is an integer selected from 1, 2 and 3.
Preferably, n is 2 or 3.
In another embodiment, n is 1 or 2.
More preferably, n is 2.

The following preferred embodiments of compounds of the formula I are described using generic formulae I.1 to 1.11, wherein any tautomers, solvates, hydrates and salts thereof, in particular the pharmaceutically acceptable salts thereof, are encompassed. R^{2a} and R^{2b} are as defined for R².

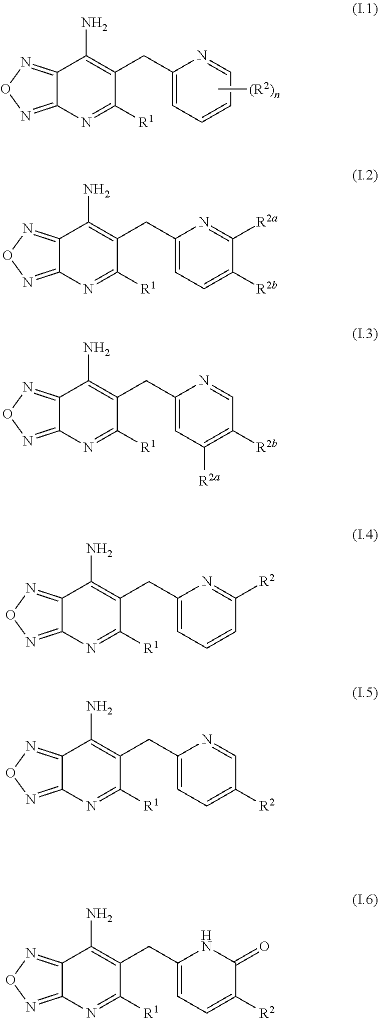

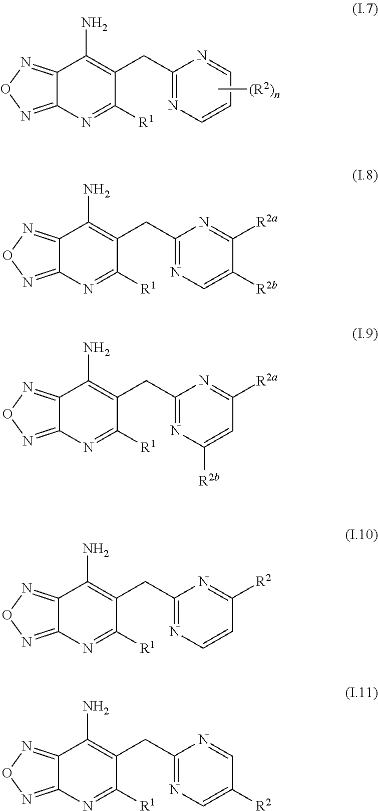

Another embodiment concerns those compounds of formula

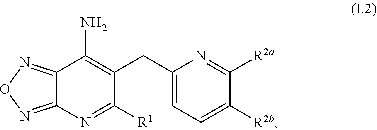

wherein
R¹ is CH₃;
R^{2a} and R^{2b} are each independently selected from the group consisting of:

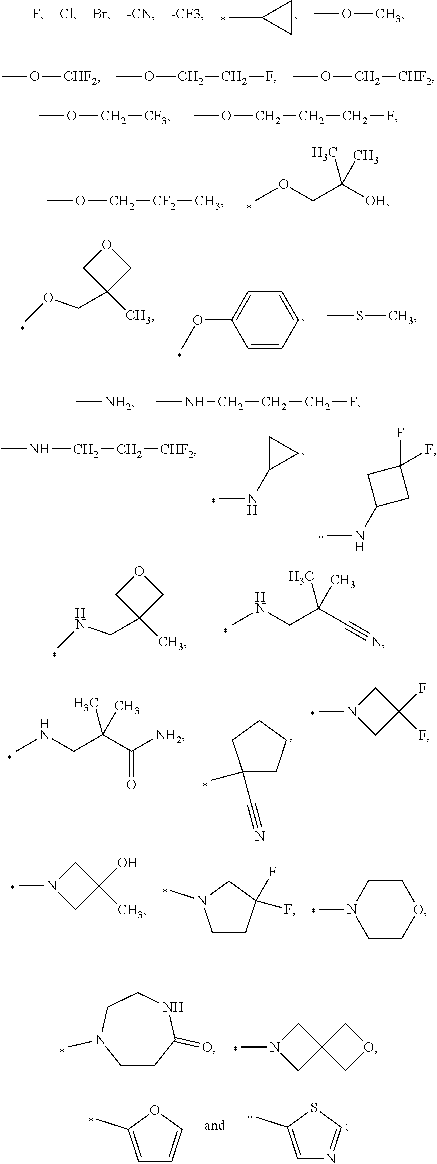

and
n is 1 or 2;
or a salt thereof, particularly a pharmaceutically acceptable salt thereof.

Compounds of formula I include:

| Ex. | |
|---|---|
| No. | Structure |
| 1 | 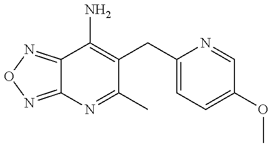 [SMILES: COc1ccc(Cc2c(C)nc3nonc3c2N)nc1] |
| 2 | 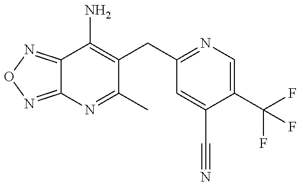 [SMILES: Cc1nc2nonc2c(N)c1Cc1cc(C#N)c(C(F)(F)F)cn1] |
| 3 | 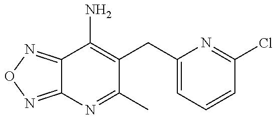 [SMILES: Cc1nc2nonc2c(N)c1Cc1cccc(Cl)n1] |
| 4 | 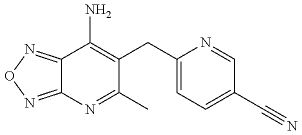 [SMILES: Cc1nc2nonc2c(N)c1Cc1ccc(C#N)cn1] |
| 5 | 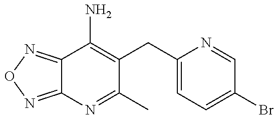 [SMILES: Cc1nc2nonc2c(N)c1Cc1ccc(Br)cn1] |
| 6 | 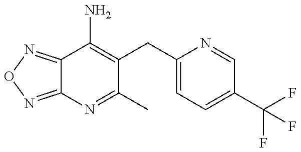 [SMILES: Cc1nc2nonc2c(N)c1Cc1ccc(C(F)(F)F)cn1] |
| 7 | 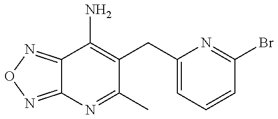 [SMILES: Cc1nc2nonc2c(N)c1Cc1cccc(Br)n1] |
| 8 | 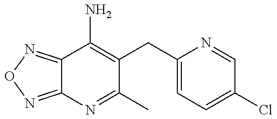 [SMILES: Cc1nc2nonc2c(N)c1Cc1ccc(Cl)cn1] |
| 9 | 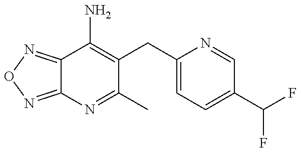 [SMILES: Cc1nc2nonc2c(N)c1Cc1ccc(C(F)F)cn1] |
| 10 | 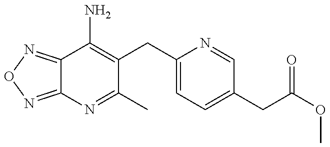 [SMILES: COC(=O)Cc1ccc(Cc2c(C)nc3nonc3c2N)nc1] |
| 11 | 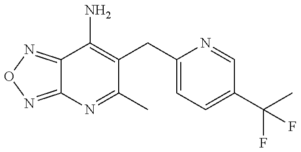 [SMILES: Cc1nc2nonc2c(N)c1Cc1ccc(C(C)(F)F)cn1] |
| 12 | 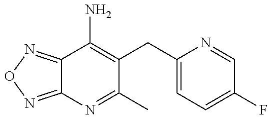 [SMILES: Cc1nc2nonc2c(N)c1Cc1ccc(F)cn1] |
| 13 | 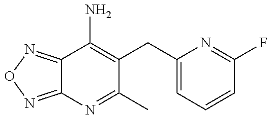 [SMILES: Cc1nc2nonc2c(N)c1Cc1cccc(F)n1] |
| 14 | 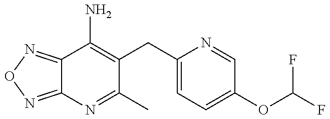 [SMILES: Cc1nc2nonc2c(N)c1Cc1ccc(OC(F)F)cn1] |
| 15 | 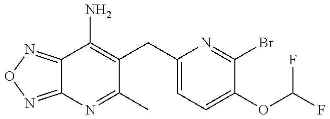 [SMILES: Cc1nc2nonc2c(N)c1Cc1ccc(OC(F)F)c(Br)n1] |
| 16 | 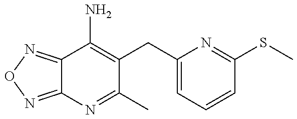 [SMILES: CSc1cccc(Cc2c(C)nc3nonc3c2N)n1] |
| 17 | 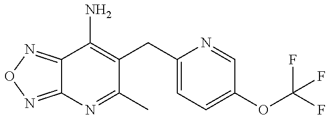 [SMILES: Cc1nc2nonc2c(N)c1Cc1ccc(OC(F)(F)F)cn1] |
| 18 | 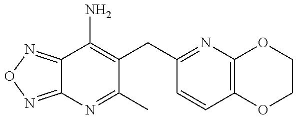 [SMILES: Cc1nc2nonc2c(N)c1Cc1ccc2c(n1)OCCO2] |
| 19 | 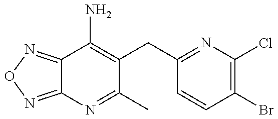 [SMILES: Cc1nc2nonc2c(N)c1Cc1ccc(Br)c(Cl)n1] |
| 20 | 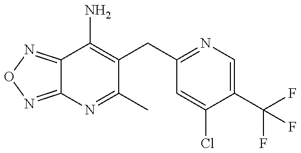 [SMILES: Cc1nc2nonc2c(N)c1Cc1cc(Cl)c(C(F)(F)F)cn1] |
| 21 | 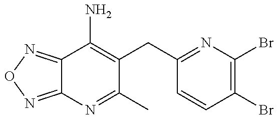 [SMILES: Cc1nc2nonc2c(N)c1Cc1ccc(Br)c(Br)n1] |
| 22 | 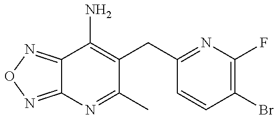 [SMILES: Cc1nc2nonc2c(N)c1Cc1ccc(Br)c(F)n1] |
| 23 | 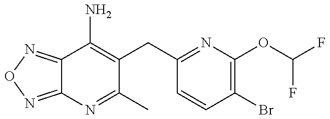 [SMILES: Cc1nc2nonc2c(N)c1Cc1ccc(Br)c(OC(F)F)n1] |
| 24 | 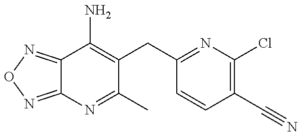 [SMILES: Cc1nc2nonc2c(N)c1Cc1ccc(C#N)c(Cl)n1] |
| 25 | 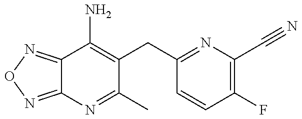 [SMILES: Cc1nc2nonc2c(N)c1Cc1ccc(F)c(C#N)n1] |
| 26 | 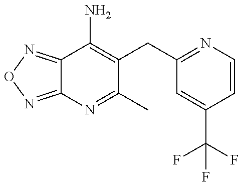 [SMILES: Cc1nc2nonc2c(N)c1Cc1cc(C(F)(F)F)ccn1] |
| 27 | 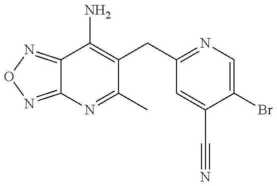 [SMILES: Cc1nc2nonc2c(N)c1Cc1cc(C#N)c(Br)cn1] |
| 28 | 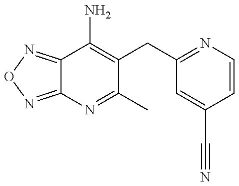 [SMILES: Cc1nc2nonc2c(N)c1Cc1cc(C#N)ccn1] |
| 29 | 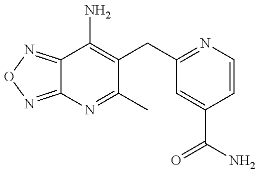 [SMILES: Cc1nc2nonc2c(N)c1Cc1cc(C(N)=O)ccn1] |
| 30 | 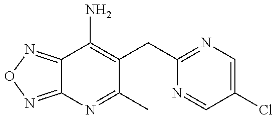 [SMILES: Cc1nc2nonc2c(N)c1Cc1ncc(Cl)cn1] |
| 31 | 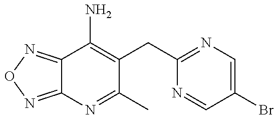 [SMILES: Cc1nc2nonc2c(N)c1Cc1ncc(Br)cn1] |
| 32 | 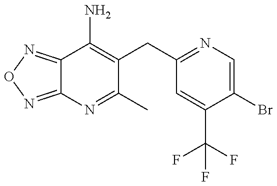 [SMILES: Cc1nc2nonc2c(N)c1Cc1cc(C(F)(F)F)c(Br)cn1] |
| 33 | 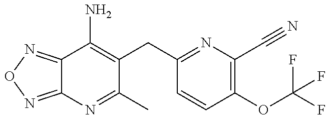 [SMILES: Cc1nc2nonc2c(N)c1Cc1ccc(OC(F)(F)F)c(C#N)n1] |
| 34 | 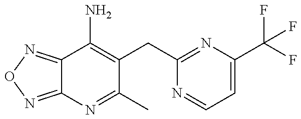 [SMILES: Cc1nc2nonc2c(N)c1Cc1nccc(C(F)(F)F)n1] |
| 35 | 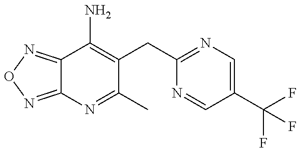 [SMILES: Cc1nc2nonc2c(N)c1Cc1ncc(C(F)(F)F)cn1] |
| 36 | 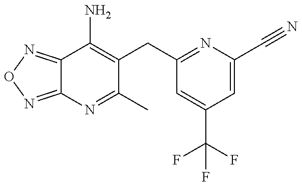 [SMILES: Cc1nc2nonc2c(N)c1Cc1cc(C(F)(F)F)cc(C#N)n1] |
| 37 | 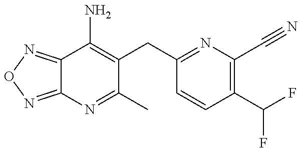 [SMILES: Cc1nc2nonc2c(N)c1Cc1ccc(C(F)F)c(C#N)n1] |
| 38 | 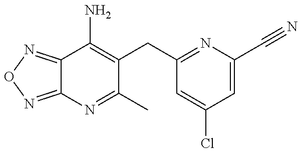 [SMILES: Cc1nc2nonc2c(N)c1Cc1cc(Cl)cc(C#N)n1] |
| 39 | 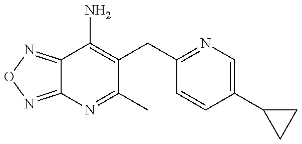 [SMILES: Cc1nc2nonc2c(N)c1Cc1ccc(C2CC2)cn1] |
| 40 | 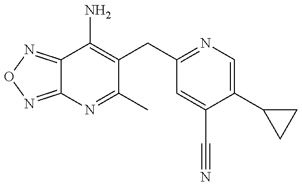 [SMILES: Cc1nc2nonc2c(N)c1Cc1cc(C#N)c(C2CC2)cn1] |
| 41 | 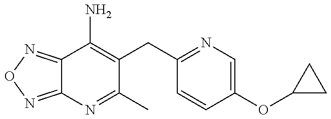 [SMILES: Cc1nc2nonc2c(N)c1Cc1ccc(OC2CC2)cn1] |
| 42 | 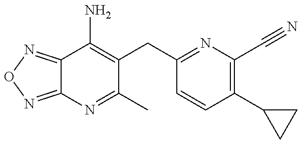 [SMILES: Cc1nc2nonc2c(N)c1Cc1ccc(C2CC2)c(C#N)n1] |
| 43 | 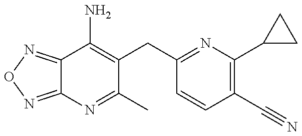 [SMILES: Cc1nc2nonc2c(N)c1Cc1ccc(C#N)c(C2CC2)n1] |
| 44 | 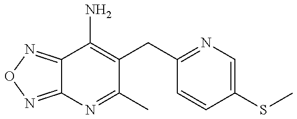 [SMILES: CSc1ccc(Cc2c(C)nc3nonc3c2N)nc1] |
| 45 | 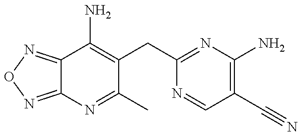 [SMILES: Cc1nc2nonc2c(N)c1Cc1ncc(C#N)c(N)n1] |
| 46 | 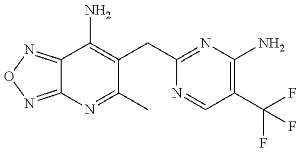 [SMILES: Cc1nc2nonc2c(N)c1Cc1ncc(C(F)(F)F)c(N)n1] |
| 47 | 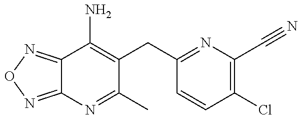 [SMILES: Cc1nc2nonc2c(N)c1Cc1ccc(Cl)c(C#N)n1] |
| 48 | 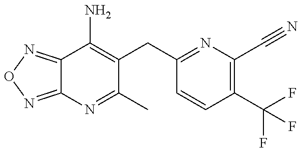 [SMILES: Cc1nc2nonc2c(N)c1Cc1ccc(C(F)(F)F)c(C#N)n1] |
| 49 | 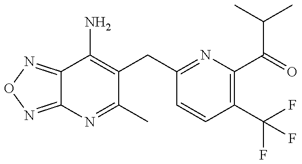 [SMILES: Cc1nc2nonc2c(N)c1Cc1ccc(C(F)(F)F)c(C(=O)C(C)C)n1] |
| 50 | 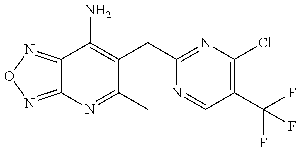 [SMILES: Cc1nc2nonc2c(N)c1Cc1ncc(C(F)(F)F)c(Cl)n1] |
| 51 | 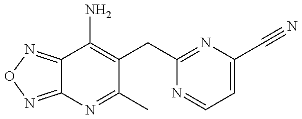 [SMILES: Cc1nc2nonc2c(N)c1Cc1nccc(C#N)n1] |
| 52 | 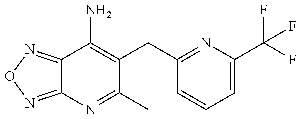 [SMILES: Cc1nc2nonc2c(N)c1Cc1cccc(C(F)(F)F)n1] |
| 53 | 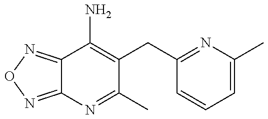 [SMILES: Cc1cccc(Cc2c(C)nc3nonc3c2N)n1] |
| 54 | 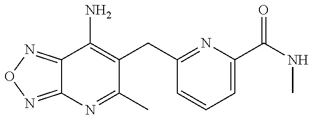 [SMILES: CNC(=O)c1cccc(Cc2c(C)nc3nonc3c2N)n1] |
| 55 | 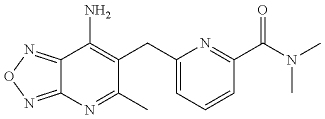 [SMILES: Cc1nc2nonc2c(N)c1Cc1cccc(C(=O)N(C)C)n1] |
| 56 | 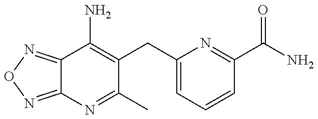 [SMILES: Cc1nc2nonc2c(N)c1Cc1cccc(C(N)=O)n1] |
| 57 | 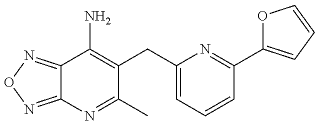 [SMILES: Cc1nc2nonc2c(N)c1Cc1cccc(-c2ccco2)n1] |
| 58 | 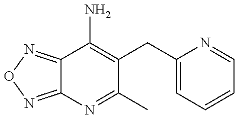 [SMILES: Cc1nc2nonc2c(N)c1Cc1ccccn1] |
| 59 | 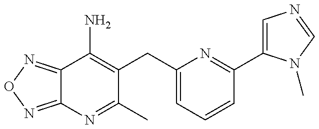 [SMILES: Cc1nc2nonc2c(N)c1Cc1cccc(-c2cncn2C)n1] |
| 60 | 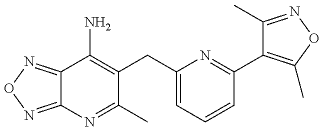 [SMILES: Cc1nc2nonc2c(N)c1Cc1cccc(-c2c(C)noc2C)n1] |
| 61 | 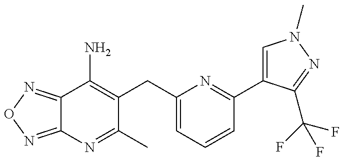 [SMILES: Cc1nc2nonc2c(N)c1Cc1cccc(-c2cn(C)nc2C(F)(F)F)n1] |
| 62 | 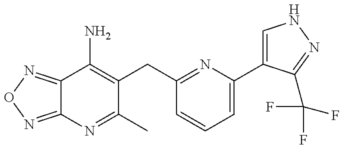 [SMILES: Cc1nc2nonc2c(N)c1Cc1cccc(-c2c[nH]nc2C(F)(F)F)n1] |
| 63 | 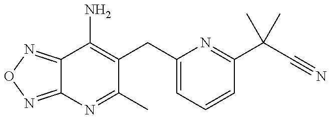 [SMILES: Cc1nc2nonc2c(N)c1Cc1cccc(C(C)(C)C#N)n1] |
| 64 | 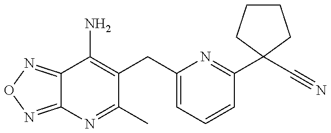 [SMILES: Cc1nc2nonc2c(N)c1Cc1cccc(C2(C#N)CCCC2)n1] |
| 65 | 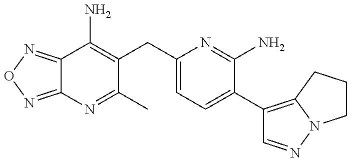 [SMILES: Cc1nc2nonc2c(N)c1Cc1ccc(-c2cnn3c2CCC3)c(N)n1] |
| 66 | 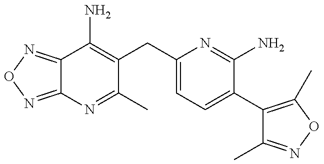 [SMILES: Cc1nc2nonc2c(N)c1Cc1ccc(-c2c(C)noc2C)c(N)n1] |
| 67 | 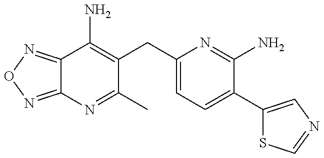 [SMILES: Cc1nc2nonc2c(N)c1Cc1ccc(-c2cncs2)c(N)n1] |
| 68 | 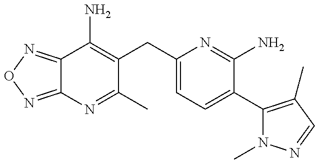 [SMILES: Cc1cnn(C)c1-c1ccc(Cc2c(C)nc3nonc3c2N)nc1N] |
| 69 | 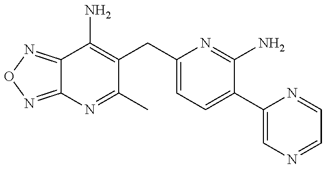 [SMILES: Cc1nc2nonc2c(N)c1Cc1ccc(-c2cnccn2)c(N)n1] |
| 70 | 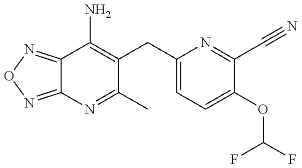 [SMILES: Cc1nc2nonc2c(N)c1Cc1ccc(OC(F)F)c(C#N)n1] |
| 71 | 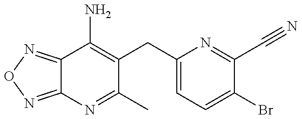 [SMILES: Cc1nc2nonc2c(N)c1Cc1ccc(Br)c(C#N)n1] |
| 72 | 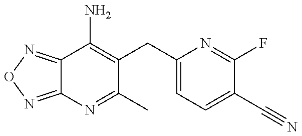 [SMILES: Cc1nc2nonc2c(N)c1Cc1ccc(C#N)c(F)n1] |
| 73 | 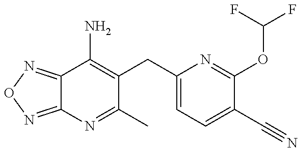 [SMILES: Cc1nc2nonc2c(N)c1Cc1ccc(C#N)c(OC(F)F)n1] |
| 74 | 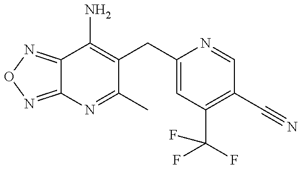 [SMILES: Cc1nc2nonc2c(N)c1Cc1cc(C(F)(F)F)c(C#N)cn1] |
| 75 | 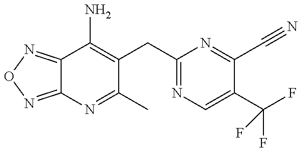 [SMILES: Cc1nc2nonc2c(N)c1Cc1ncc(C(F)(F)F)c(C#N)n1] |
| 76 | 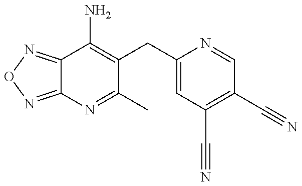 [SMILES: Cc1nc2nonc2c(N)c1Cc1cc(C#N)c(C#N)cn1] |
| 77 | 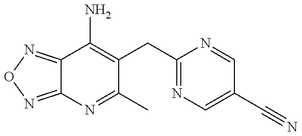 [SMILES: Cc1nc2nonc2c(N)c1Cc1ncc(C#N)cn1] |
| 78 | 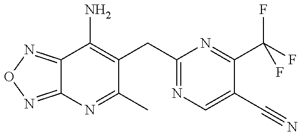 [SMILES: Cc1nc2nonc2c(N)c1Cc1ncc(C#N)c(C(F)(F)F)n1] |
| 79 | 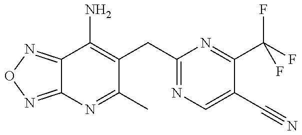 [SMILES: Cc1nc2nonc2c(N)c1Cc1ncc(C#N)c(C(F)(F)F)n1] |
| 80 | 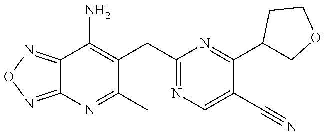 [SMILES: Cc1nc2nonc2c(N)c1Cc1ncc(C#N)c(C2CCOC2)n1] |
| 81 | 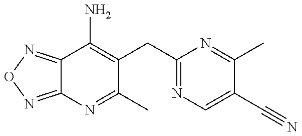 [SMILES: Cc1nc(Cc2c(C)nc3nonc3c2N)ncc1C#N] |
| 82 | 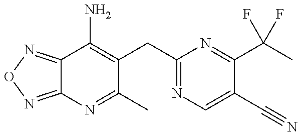 [SMILES: Cc1nc2nonc2c(N)c1Cc1ncc(C#N)c(C(C)(F)F)n1] |
| 83 | 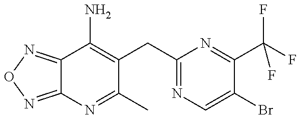 [SMILES: Cc1nc2nonc2c(N)c1Cc1ncc(Br)c(C(F)(F)F)n1] |
| 84 | 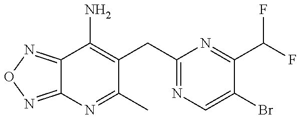 [SMILES: Cc1nc2nonc2c(N)c1Cc1ncc(Br)c(C(F)F)n1] |
| 85 | 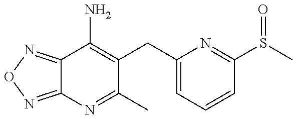 [SMILES: Cc1nc2nonc2c(N)c1Cc1cccc(S(C)=O)n1] |
| 86 | 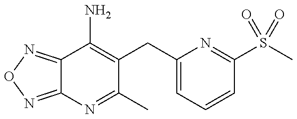 [SMILES: Cc1nc2nonc2c(N)c1Cc1cccc(S(C)(=O)=O)n1] |
| 87 | 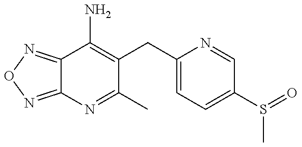 [SMILES: Cc1nc2nonc2c(N)c1Cc1ccc(S(C)=O)cn1] |
| 88 | 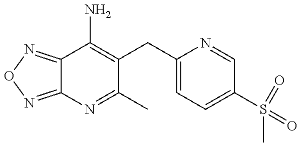 [SMILES: Cc1nc2nonc2c(N)c1Cc1ccc(S(C)(=O)=O)cn1] |
| 89 | 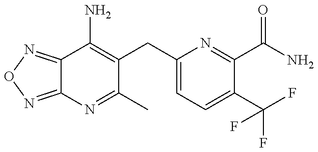 [SMILES: Cc1nc2nonc2c(N)c1Cc1ccc(C(F)(F)F)c(C(N)=O)n1] |
| 90 | 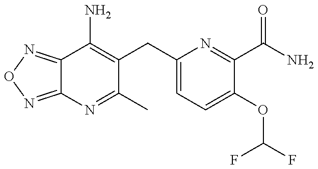 [SMILES: Cc1nc2nonc2c(N)c1Cc1ccc(OC(F)F)c(C(N)=O)n1] |
| 91 | 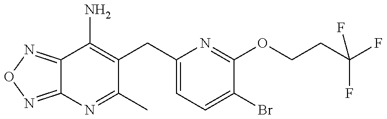 [SMILES: Cc1nc2nonc2c(N)c1Cc1ccc(Br)c(OCCC(F)(F)F)n1] |
| 92 | 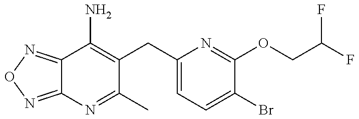 [SMILES: Cc1nc2nonc2c(N)c1Cc1ccc(Br)c(OCC(F)F)n1] |
| 93 | 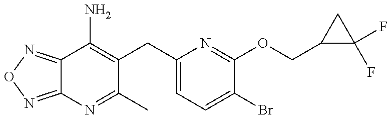 [SMILES: Cc1nc2nonc2c(N)c1Cc1ccc(Br)c(OCC2CC2(F)F)n1] |
| 94 | 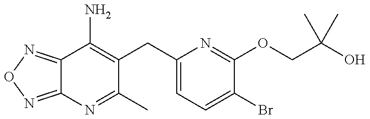 [SMILES: Cc1nc2nonc2c(N)c1Cc1ccc(Br)c(OCC(C)(C)O)n1] |
| 95 | 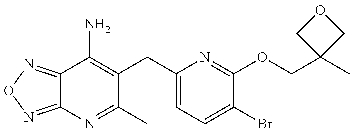 [SMILES: Cc1nc2nonc2c(N)c1Cc1ccc(Br)c(OCC2(C)COC2)n1] |
| 96 | 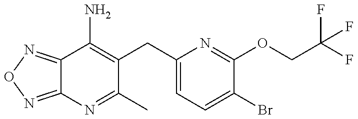 [SMILES: Cc1nc2nonc2c(N)c1Cc1ccc(Br)c(OCC(F)(F)F)n1] |
| 97 | 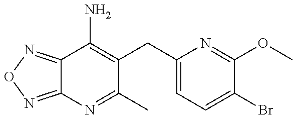 [SMILES: COc1nc(Cc2c(C)nc3nonc3c2N)ccc1Br] |
| 98 | 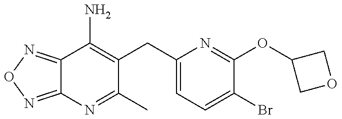 [SMILES: Cc1nc2nonc2c(N)c1Cc1ccc(Br)c(OC2COC2)n1] |
| 99 | 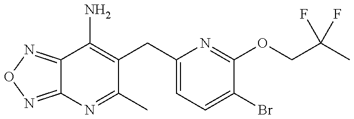 [SMILES: Cc1nc2nonc2c(N)c1Cc1ccc(Br)c(OCC(C)(F)F)n1] |
| 100 | 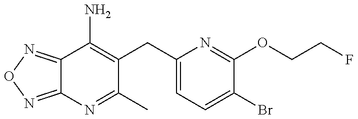 [SMILES: Cc1nc2nonc2c(N)c1Cc1ccc(Br)c(OCCF)n1] |
| 101 | 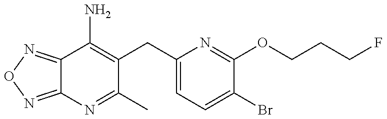 [SMILES: Cc1nc2nonc2c(N)c1Cc1ccc(Br)c(OCCCF)n1] |
| 102 | 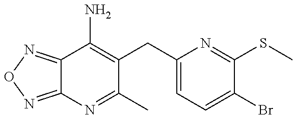 [SMILES: CSc1nc(Cc2c(C)nc3nonc3c2N)ccc1Br] |
| 103 | 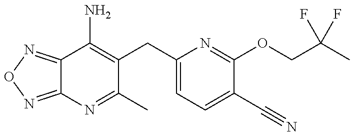 [SMILES: Cc1nc2nonc2c(N)c1Cc1ccc(C#N)c(OCC(C)(F)F)n1] |
| 104 | 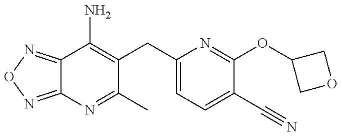 [SMILES: Cc1nc2nonc2c(N)c1Cc1ccc(C#N)c(OC2COC2)n1] |
| 105 | 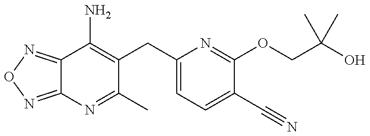 [SMILES: Cc1nc2nonc2c(N)c1Cc1ccc(C#N)c(OCC(C)(C)O)n1] |
| 106 | 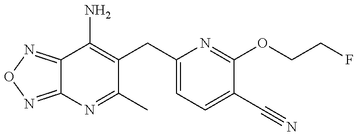 [SMILES: Cc1nc2nonc2c(N)c1Cc1ccc(C#N)c(OCCF)n1] |
| 107 | 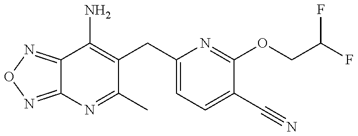 [SMILES: Cc1nc2nonc2c(N)c1Cc1ccc(C#N)c(OCC(F)F)n1] |
| 108 | 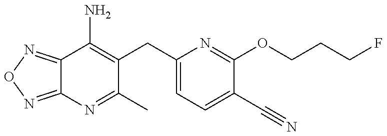 [SMILES: Cc1nc2nonc2c(N)c1Cc1ccc(C#N)c(OCCCF)n1] |
| 109 | 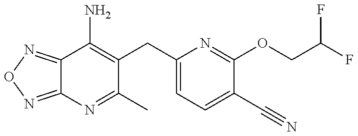 [SMILES: Cc1nc2nonc2c(N)c1Cc1ccc(C#N)c(OCC(F)F)n1] |
| 110 | 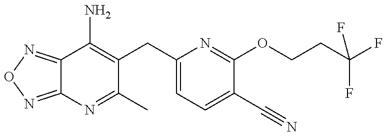 [SMILES: Cc1nc2nonc2c(N)c1Cc1ccc(C#N)c(OCCC(F)(F)F)n1] |
| 111 | 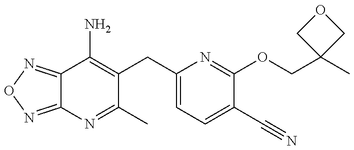 [SMILES: Cc1nc2nonc2c(N)c1Cc1ccc(C#N)c(OCC2(C)COC2)n1] |
| 112 | 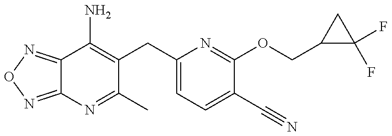 [SMILES: Cc1nc2nonc2c(N)c1Cc1ccc(C#N)c(OCC2CC2(F)F)n1] |
| 113 | 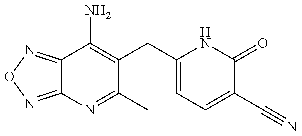 [SMILES: Cc1nc2nonc2c(N)c1Cc1ccc(C#N)c(=O)[nH]1] |
| 114 | 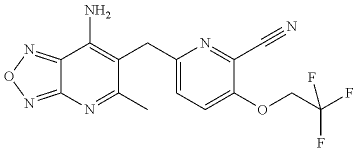 [SMILES: Cc1nc2nonc2c(N)c1Cc1ccc(OCC(F)(F)F)c(C#N)n1] |
| 115 | 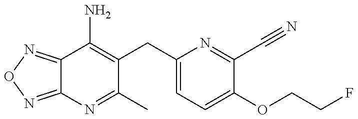 [SMILES: Cc1nc2nonc2c(N)c1Cc1ccc(OCCF)c(C#N)n1] |
| 116 | 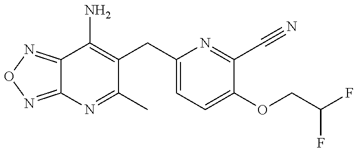 [SMILES: Cc1nc2nonc2c(N)c1Cc1ccc(OCC(F)F)c(C#N)n1] |
| 117 | 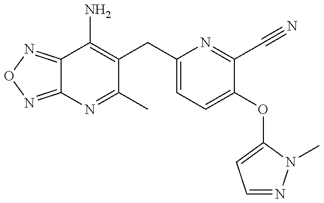 [SMILES: Cc1nc2nonc2c(N)c1Cc1ccc(Oc2ccnn2C)c(C#N)n1] |
| 118 | 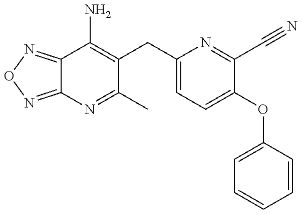 [SMILES: Cc1nc2nonc2c(N)c1Cc1ccc(Oc2ccccc2)c(C#N)n1] |
| 119 | 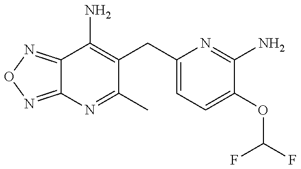 [SMILES: Cc1nc2nonc2c(N)c1Cc1ccc(OC(F)F)c(N)n1] |
| 120 | 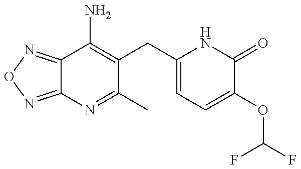 [SMILES: Cc1nc2nonc2c(N)c1Cc1ccc(OC(F)F)c(=O)[nH]1] |
| 121 | 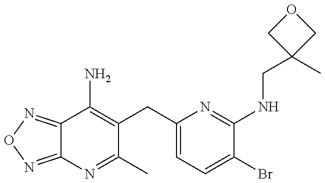 [SMILES: Cc1nc2nonc2c(N)c1Cc1ccc(Br)c(NCC2(C)COC2)n1] |
| 122 | 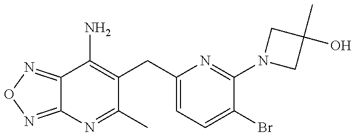 [SMILES: Cc1nc2nonc2c(N)c1Cc1ccc(Br)c(N2CC(C)(O)C2)n1] |
| 123 | 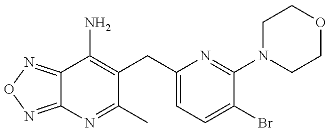 [SMILES: Cc1nc2nonc2c(N)c1Cc1ccc(Br)c(N2CCOCC2)n1] |
| 124 | 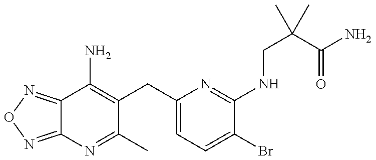 [SMILES: Cc1nc2nonc2c(N)c1Cc1ccc(Br)c(NCC(C)(C)C(N)=O)n1] |
| 125 | 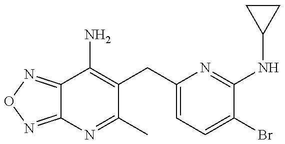 [SMILES: Cc1nc2nonc2c(N)c1Cc1ccc(Br)c(NC2CC2)n1] |
| 126 | 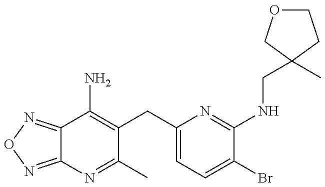 [SMILES: Cc1nc2nonc2c(N)c1Cc1ccc(Br)c(NCC2(C)CCOC2)n1] |
| 127 | 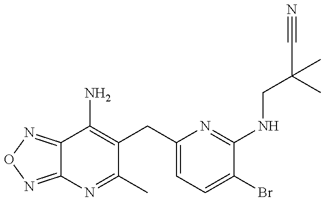 [SMILES: Cc1nc2nonc2c(N)c1Cc1ccc(Br)c(NCC(C)(C)C#N)n1] |
| 128 | 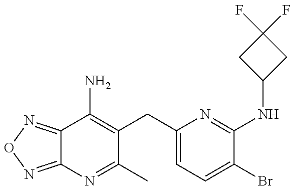 [SMILES: Cc1nc2nonc2c(N)c1Cc1ccc(Br)c(NC2CC(F)(F)C2)n1] |
| 129 | 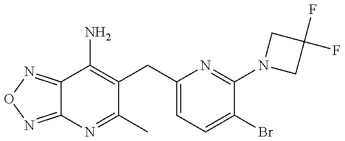 [SMILES: Cc1nc2nonc2c(N)c1Cc1ccc(Br)c(N2CC(F)(F)C2)n1] |
| 130 | 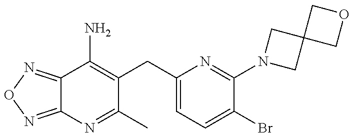 [SMILES: Cc1nc2nonc2c(N)c1Cc1ccc(Br)c(N2CC3(COC3)C2)n1] |
| 131 | 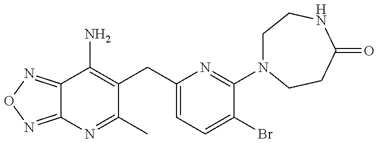 [SMILES: Cc1nc2nonc2c(N)c1Cc1ccc(Br)c(N2CCNC(=O)CC2)n1] |
| 132 | 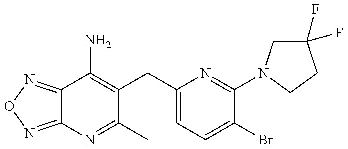 [SMILES: Cc1nc2nonc2c(N)c1Cc1ccc(Br)c(N2CCC(F)(F)C2)n1] |
| 133 | 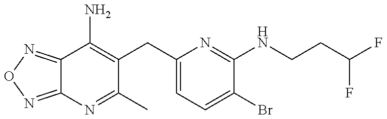 [SMILES: Cc1nc2nonc2c(N)c1Cc1ccc(Br)c(NCCC(F)F)n1] |
| 134 | 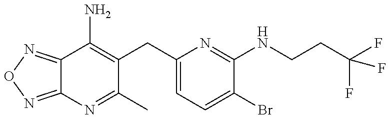 [SMILES: Cc1nc2nonc2c(N)c1Cc1ccc(Br)c(NCCC(F)(F)F)n1] |
| 135 | 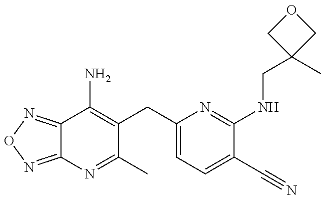 [SMILES: Cc1nc2nonc2c(N)c1Cc1ccc(C#N)c(NCC2(C)COC2)n1] |
| 136 | 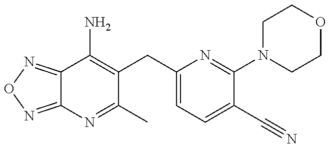 [SMILES: Cc1nc2nonc2c(N)c1Cc1ccc(C#N)c(N2CCOCC2)n1] |
| 137 | 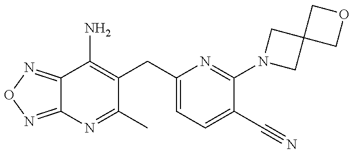 [SMILES: Cc1nc2nonc2c(N)c1Cc1ccc(C#N)c(N2CC3(COC3)C2)n1] |
| 138 | 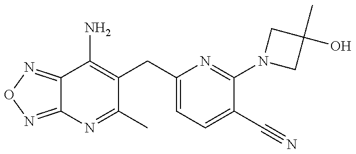 [SMILES: Cc1nc2nonc2c(N)c1Cc1ccc(C#N)c(N2CC(C)(O)C2)n1] |
| 139 | 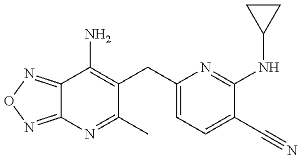 [SMILES: Cc1nc2nonc2c(N)c1Cc1ccc(C#N)c(NC2CC2)n1] |
| 140 | 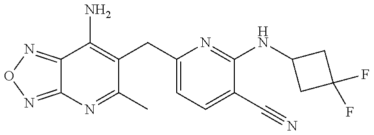 [SMILES: Cc1nc2nonc2c(N)c1Cc1ccc(C#N)c(NC2CC(F)(F)C2)n1] |
| 141 | 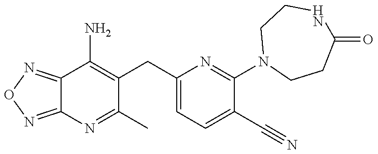 [SMILES: Cc1nc2nonc2c(N)c1Cc1ccc(C#N)c(N2CCNC(=O)CC2)n1] |
| 142 | 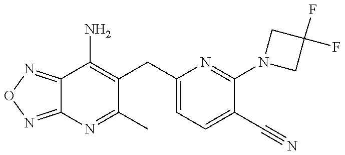 [SMILES: Cc1nc2nonc2c(N)c1Cc1ccc(C#N)c(N2CC(F)(F)C2)n1] |
| 143 | 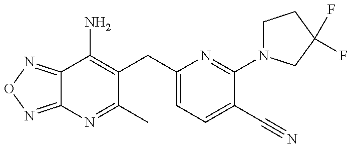 [SMILES: Cc1nc2nonc2c(N)c1Cc1ccc(C#N)c(N2CCC(F)(F)C2)n1] |
| 144 | 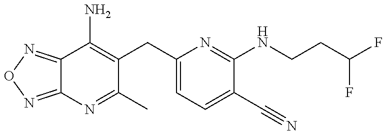 [SMILES: Cc1nc2nonc2c(N)c1Cc1ccc(C#N)c(NCCC(F)F)n1] |
| 145 | 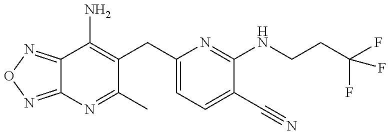 [SMILES: Cc1nc2nonc2c(N)c1Cc1ccc(C#N)c(NCCC(F)(F)F)n1] |
| 146 | 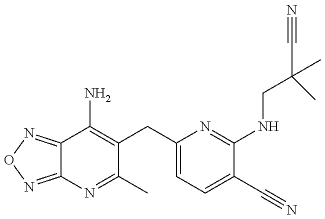 [SMILES: Cc1nc2nonc2c(N)c1Cc1ccc(C#N)c(NCC(C)(C)C#N)n1] |
| 147 | 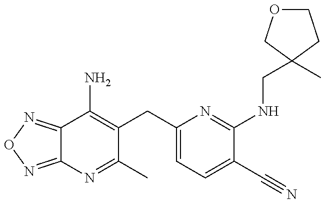 [SMILES: Cc1nc2nonc2c(N)c1Cc1ccc(C#N)c(NCC2(C)CCOC2)n1] |
| 148 | 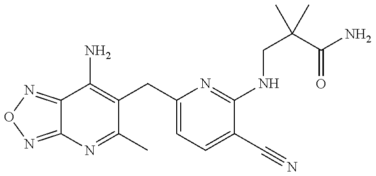 [SMILES: Cc1nc2nonc2c(N)c1Cc1ccc(C#N)c(NCC(C)(C)C(N)=O)n1] |
| 149 | 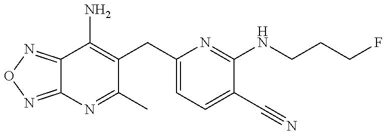 [SMILES: Cc1nc2nonc2c(N)c1Cc1ccc(C#N)c(NCCCF)n1] |
| 150 | 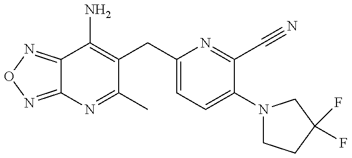 [SMILES: Cc1nc2nonc2c(N)c1Cc1ccc(N2CCC(F)(F)C2)c(C#N)n1] |
| 151 | 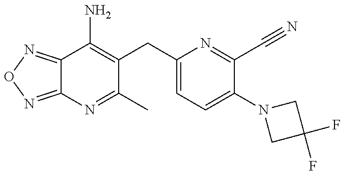 [SMILES: Cc1nc2nonc2c(N)c1Cc1ccc(N2CC(F)(F)C2)c(C#N)n1] |
| 152 | 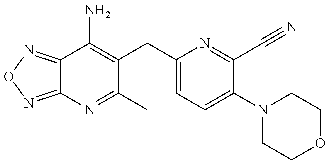 [SMILES: Cc1nc2nonc2c(N)c1Cc1ccc(N2CCOCC2)c(C#N)n1] |
| 153 | 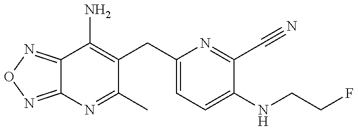 [SMILES: Cc1nc2nonc2c(N)c1Cc1ccc(NCCF)c(C#N)n1] |
| 154 | 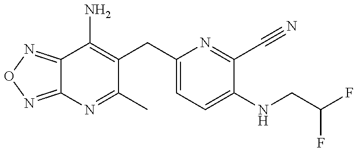 [SMILES: Cc1nc2nonc2c(N)c1Cc1ccc(NCC(F)F)c(C#N)n1] |
| 155 | 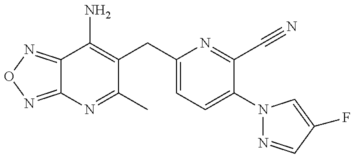 [SMILES: Cc1nc2nonc2c(N)c1Cc1ccc(-n2cc(F)cn2)c(C#N)n1] |
| 156 | 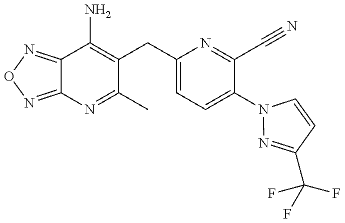 [SMILES: Cc1nc2nonc2c(N)c1Cc1ccc(-n2ccc(C(F)(F)F)n2)c(C#N)n1] |
| 157 | 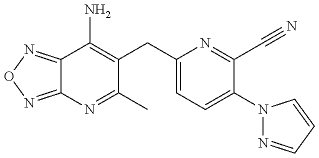 [SMILES: Cc1nc2nonc2c(N)c1Cc1ccc(-n2cccn2)c(C#N)n1] |
| 158 | 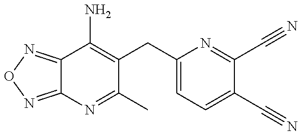 [SMILES: Cc1nc2nonc2c(N)c1Cc1ccc(C#N)c(C#N)n1] |
| 159 | 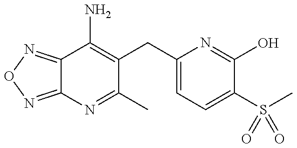 [SMILES: Cc1nc2nonc2c(N)c1Cc1ccc(S(C)(=O)=O)c(O)n1] |
| 160 | 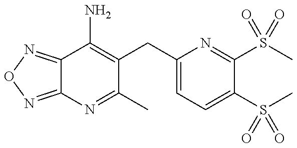 [SMILES: Cc1nc2nonc2c(N)c1Cc1ccc(S(C)(=O)=O)c(S(C)(=O)=O)n1] |
| 161 | 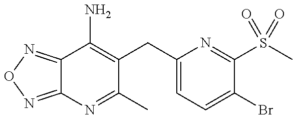 [SMILES: Cc1nc2nonc2c(N)c1Cc1ccc(Br)c(S(C)(=O)=O)n1] |
| 162 | 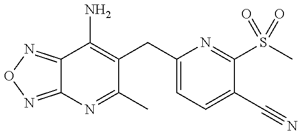 [SMILES: Cc1nc2nonc2c(N)c1Cc1ccc(C#N)c(S(C)(=O)=O)n1] |
| 163 | 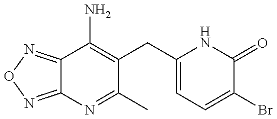 [SMILES: Cc1nc2nonc2c(N)c1Cc1ccc(Br)c(=O)[nH]1] |
| 164 | 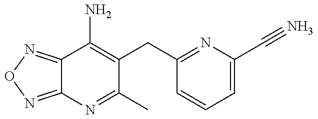 [SMILES: Cc1nc2nonc2c(N)c1Cc1cccc(C#N)n1] |
| 165 | 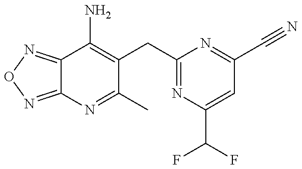 [SMILES: Cc1nc2nonc2c(N)c1Cc1nc(C#N)cc(C(F)F)n1] |
| 166 | 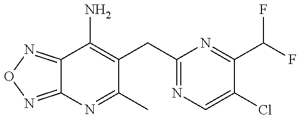 [SMILES: Cc1nc2nonc2c(N)c1Cc1ncc(Cl)c(C(F)F)n1] |
| 167 | 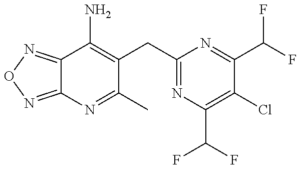 [SMILES: Cc1nc2nonc2c(N)c1Cc1nc(C(F)F)c(Cl)c(C(F)F)n1] |

Preferred compounds of formula I include:

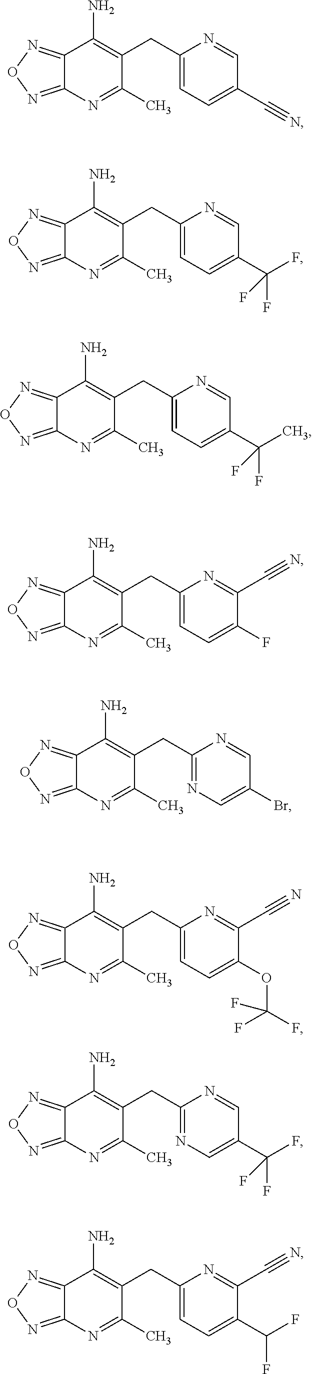

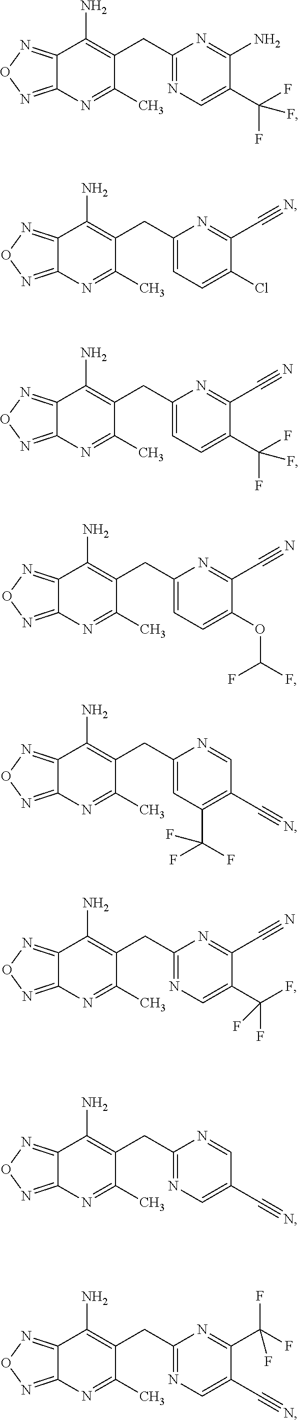

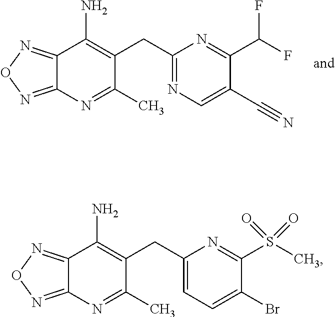 [SMILES: Cc1nc2nonc2c(N)c1Cc1ncc(C#N)c(C(F)F)n1 ; Cc1nc2nonc2c(N)c1Cc1ccc(Br)c(S(C)(=O)=O)n1]

or a salt thereof, particularly a pharmaceutically acceptable salt thereof.

Particularly preferred compounds of formula I, including their tautomers and stereoisomers, the salts thereof, or any solvates or hydrates thereof, are described in the experimental section hereinafter.

The compounds according to the invention and their intermediates may be obtained using methods of synthesis which are known to the one skilled in the art and described in the literature of organic synthesis for example, as described in WO 2018/024653.

Further modifications of compounds of formula I by methods known in the art and illustrated in the Examples below, may be used to prepare additional compounds of the invention.

The compounds of general formula I may be resolved into their enantiomers and/or diastereomers as mentioned below. Thus, for example, cis/trans mixtures may be resolved into their cis and trans isomers and racemic compounds may be separated into their enantiomers.

The cis/trans mixtures may be resolved, for example, by chromatography into the cis and trans isomers thereof. The compounds of general formula I which occur as racemates may be separated by methods known per se into their optical antipodes and diastereomeric mixtures of compounds of general formula I may be resolved into their diastereomers by taking advantage of their different physico-chemical properties using methods known per se, e.g. chromatography and/or fractional crystallization; if the compounds obtained thereafter are racemates, they may be resolved into the enantiomers as mentioned below.

The racemates are preferably resolved by column chromatography on chiral phases or by crystallization from an optically active solvent or by reacting with an optically active substance which forms salts or derivatives such as esters or amides with the racemic compound. Salts may be formed with enantiomerically pure acids for basic compounds and with enantiomerically pure bases for acidic compounds. Diastereomeric derivatives are formed with enantiomerically pure auxiliary compounds, e.g. acids, their activated derivatives, or alcohols. Separation of the diastereomeric mixture of salts or derivatives thus obtained may be achieved by taking advantage of their different physico-chemical properties, e.g. differences in solubility; the free antipodes may be released from the pure diastereomeric salts or derivatives by the action of suitable agents. Optically active acids commonly used for such a purpose as well as optically active alcohols applicable as auxiliary residues are known to those skilled in the art.

As mentioned above, the compounds of formula I may be converted into salts, particularly for pharmaceutical use into the pharmaceutically acceptable salts. As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like.

For example, such salts include salts from benzenesulfonic acid, benzoic acid, citric acid, ethanesulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid.

Further pharmaceutically acceptable salts can be formed with cations from ammonia, L-arginine, calcium, 2,2′-iminobisethanol, L-lysine, magnesium, N-methyl-D-glucamine, potassium, sodium and tris(hydroxymethyl)-aminomethane.

### Terms and Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to.

The term “substance use disorder”, “SUD” and the like refers to a mental disorder that affects a person's brain and behavior, leading to their inability to control their use of substances such as legal or illegal drugs, alcohol, or medications. Substance use disorder (SUD) is also called substance addiction or substance dependence.

The term “opioid use disorder”, “OUD” and the like refers to a disease in which people continue to use opioids in spite of harms caused by their use. The term “opioid” includes drugs acting on opioid receptors, such as morphine, codeine, hydrocodone, oxycodone, fentanyl and others. Opioid use disorder (OUD) is also called opioid addiction or opioid dependence.

The term “stimulant use disorder”, “StimUD” and the like refers to a disease in which people continue to use stimulants in spite of harms caused by their use. The term “stimulant” includes cocaine, amphetamine, methamphetamine and similar drugs. Stimulant use disorder (StimUD) is also called stimulant addiction or stimulant dependence.

The term “alcohol use disorder” and the like refers to a disease in which people continue to use alcohol in spite of harms caused by their use. Alcohol use disorder is also called alcohol dependence or alcohol addiction.

The term “patients with high impulsivity” includes patients with moderate to severe impulsivity and means patients who show above average impulsivity in tasks and scales such as DDT, 4-CSRTT, BIS-11, and S—UPPS—P, or are included in this patient group based on clinical judgement. Preferably, the term “patients with moderate to severe impulsivity” means patients who show an impulsivity in tasks and scales such as DDT, 4-CSRTT, BIS-11, and S—UPPS-P that is significantly above average. Significantly above average means impulsivity test result in the top 40% of the tested patients. A more preferred patient group consists of the patients that have impulsivity tests results within the top 30% of the tested patients.

Even more preferably, SUD patients including OUD and StimUD patients with moderate to severe impulsivity scores are those patients that prior to the treatment according to the invention, have a baseline k parameter ≥0.025 on the behavioral version of the DDT and/or at least 3 premature responses (≥3) on the short version of the 4 CSRTT. A preferred patient group for the medical treatment according to the invention have a baseline k parameter ≥0.025 on the behavioral version of the DDT.

In another embodiment, SUD patients including OUD and StimUD patients with moderate to severe impulsivity scores are those patients that prior to the treatment according to the invention, have a BIS-11 total score ≥70. This is another preferred patient group for the medical treatment according to the invention.

The term “inhibitors of the ghrelin O-acyl transferase (GOAT)” as used herein refers to compounds which have an inhibiting effect on ghrelin O-acyl transferase (GOAT) in vitro and/or in vivo. Preferably, the compounds used in the treatment according to the invention possess suitable pharmacological and pharmacokinetic properties to use them as medicaments.

The terms “compound(s) according to this invention”, “compound(s) of formula (I)”, “compound(s) of the invention” and the like denote the compounds of the formula (I) according to the present invention including their tautomers, stereoisomers and mixtures thereof and the salts thereof, in particular the pharmaceutically acceptable salts thereof, and the solvates and hydrates of such compounds, including the solvates and hydrates of such tautomers, stereoisomers and salts thereof. Preferred compounds of formula I include each of the specific compounds of examples 1 to 167, particularly the compound of example 70.

The terms “treatment” and “treating” embrace both preventative, i.e. prophylactic, or therapeutic, i.e. curative and/or palliative, treatment. Thus the terms “treatment” and “treating” comprise therapeutic treatment of patients having already developed said condition, in particular in manifest form. Therapeutic treatment may be symptomatic treatment in order to relieve the symptoms of the specific indication or causal treatment in order to reverse or partially reverse the conditions of the indication or to stop or slow down progression of the disease. Thus the compositions and methods of the present invention may be used for instance as therapeutic treatment over a period of time as well as for chronic therapy. In addition the terms “treatment” and “treating” comprise prophylactic treatment, i.e. a treatment of patients at risk to develop a condition mentioned hereinbefore, thus reducing said risk.

When this invention refers to patients requiring treatment, it relates primarily to treatment in mammals, in particular humans.

The term “therapeutically effective amount” means an amount of a compound of the present invention that (i) treats or prevents the particular disease or condition, (ii) attenuates, ameliorates, or eliminates one or more symptoms of the particular disease or condition, or (iii) prevents or delays the onset of one or more symptoms of the particular disease or condition described herein.

The terms “modulated” or “modulating”, or “modulate(s)”, as used herein, unless otherwise indicated, refer to the inhibition of the ghrelin O-acyl transferase (GOAT) with one or more compounds of the present invention.

The terms “mediated” or “mediating” or “mediate”, as used herein, unless otherwise indicated, refer to the (i) treatment, including prevention of the particular disease or condition, (ii) attenuation, amelioration, or elimination of one or more symptoms of the particular disease or condition, or (iii) prevention or delay of the onset of one or more symptoms of the particular disease or condition described herein.

The term “substituted” as used herein, means that any one or more hydrogens on the designated atom, radical or moiety is replaced with a selection from the indicated group, provided that the atom's normal valence is not exceeded, and that the substitution results in an acceptably stable compound.

In the groups, radicals, or moieties defined below, the number of carbon atoms is often specified preceding the group, for example, C₁₋₆-alkyl means an alkyl group or radical having 1 to 6 carbon atoms. In general, for groups comprising two or more subgroups, the last named subgroup is the radical attachment point, for example, the substituent “aryl-C₁₋₃-alkyl-” means an aryl group which is bound to a C₁₋₃-alkyl-group, the latter of which is bound to the core or to the group to which the substituent is attached.

In case a compound of the present invention is depicted in form of a chemical name and as a formula in case of any discrepancy the formula shall prevail.

An asterisk may be used in sub-formulas to indicate the bond which is connected to the core molecule as defined.

The numeration of the atoms of a substituent starts with the atom which is closest to the core or to the group to which the substituent is attached.

For example, the term “3-carboxypropyl-group” represents the following substituent:

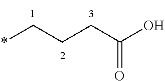 [SMILES: *CCCC(=O)O]

wherein the carboxy group is attached to the third carbon atom of the propyl group. The terms “1-methylpropyl-”, “2,2-dimethylpropyl-” or “cyclopropylmethyl-” group represent the following groups:

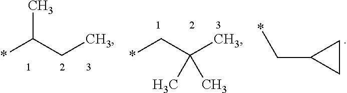

The asterisk may be used in sub-formulas to indicate the bond which is connected to the core molecule as defined.

In a definition of a group the term “wherein each X, Y and Z group is optionally substituted with” and the like denotes that each group X, each group Y and each group Z either each as a separate group or each as part of a composed group may be substituted as defined. For example a definition “R^{ex} denotes H, C₁₋₃-alkyl, C₃₋₆-cycloalkyl, C₃₋₆-cycloalkyl-C₁₋₃-alkyl or C₁₋₃-alkyl-O—, wherein each alkyl group is optionally substituted with one or more L^{ex}.” or the like means that in each of the beforementioned groups which comprise the term alkyl, i.e. in each of the groups C₁₋₃-alkyl, C₃₋₆-cycloalkyl-C₁₋₃-alkyl and C₁₋₃-alkyl-O—, the alkyl moiety may be substituted with L^{ex} as defined.

Unless specifically indicated, throughout the specification and the appended claims, a given chemical formula or name shall encompass tautomers and all stereo, optical and geometrical isomers (e.g. enantiomers, diastereomers, E/Z isomers etc.,) and racemates thereof as well as mixtures in different proportions of the separate enantiomers, mixtures of diastereomers, or mixtures of any of the foregoing forms where such isomers and enantiomers exist, as well as salts, including pharmaceutically acceptable salts thereof and solvates thereof such as for instance hydrates including solvates of the free compounds or solvates of a salt of the compound.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, and commensurate with a reasonable benefit/risk ratio.

As used herein, “pharmaceutically acceptable salts” refer to derivatives of the disclosed compounds wherein the parent compound is modified by making acid or base salts thereof.

Salts of other acids than those mentioned above which for example are useful for purifying or isolating the compounds of the present invention (e.g. trifluoro acetate salts) also comprise a part of the invention.

The term halogen generally denotes fluorine, chlorine, bromine and iodine.

The term “C₁₋ₙ-alkyl”, wherein n is an integer from 1 to n, either alone or in combination with another radical denotes an acyclic, saturated, branched or linear hydrocarbon radical with 1 to n C atoms. For example the term C₁₋₅-alkyl embraces the radicals H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂—, H₃C-C(CH₃)₂—, H₃C—CH₂—CH₂—CH₂—CH₂—, H₃C—CH₂—CH₂—CH(CH₃)—, H₃C—CH₂—CH(CH₃)—CH₂—, H₃C—CH(CH₃)—CH₂—CH₂—, H₃C—CH₂-C(CH₃)₂—, H₃C—C(CH₃)₂—CH₂—, H₃C—CH(CH₃)—CH(CH₃)— and H₃C—CH₂—CH(CH₂CH₃)—.

The term “C₁₋ₙ-alkylene” wherein n is an integer 1 to n, either alone or in combination with another radical, denotes an acyclic, straight or branched chain divalent alkyl radical containing from 1 to n carbon atoms. For example the term C₁₋₄-alkylene includes—(CH₂)—, —(CH₂—CH₂)—, —(CH(CH₃))—, —(CH₂—CH₂—CH₂)—, —(C(CH₃)₂)—, —(CH(CH₂CH₃))—, —(CH(CH₃)—CH₂)—, —(CH₂—CH(CH₃))—, —(CH₂—CH₂—CH₂—CH₂)—, —(CH₂—CH₂—CH(CH₃))—, —(CH(CH₃)—CH₂—CH₂)—, —(CH₂—CH(CH₃)—CH₂)—, —(CH₂-C(CH₃)₂)—, —(C (CH₃)₂—CH₂)—, —(CH(CH₃)—CH(CH₃))—, —(CH₂—CH(CH₂CH₃))—, —(CH(CH₂CH₃)—CH₂)—, —(CH(CH₂CH₂CH₃))—, —(CHCH(CH₃)₂)— and —C(CH₃)(CH₂CH₃)—.

The term “C₂₋ₙ-alkenyl”, is used for a group as defined in the definition for “C₁₋ₙ-alkyl” with at least two carbon atoms, if at least two of those carbon atoms of said group are bonded to each other by a double bond. For example the term C₂₋₃-alkenyl includes —CH═CH₂, —CH═CH—CH₃, —CH₂—CH═CH₂.

The term “C₂₋ₙ-alkynyl”, is used for a group as defined in the definition for “C₁₋ₙ-alkyl” with at least two carbon atoms, if at least two of those carbon atoms of said group are bonded to each other by a triple bond. For example the term C₂₋₃-alkynyl includes —C≡CH, —C≡C—CH₃, —CH₂-C≡CH.

The term “C₃-n-cycloalkyl”, wherein n is an integer 4 to n, either alone or in combination with another radical denotes a cyclic, saturated, unbranched hydrocarbon radical with 3 to n C atoms. The cyclic group may be mono-, bi-, tri- or spirocyclic, most preferably monocyclic. Examples of such cycloalkyl groups include cyclopropyl, cyclobutyl, cyclo-pentyl, cyclohexyl, cycloheptyl, cyclooctyl, cyclononyl, cyclododecyl, bicyclo[3.2.1.]oc-tyl, spiro[4.5]decyl, norpinyl, norbonyl, norcaryl, adamantyl, etc.

Many of the terms given above may be used repeatedly in the definition of a formula or group and in each case have one of the meanings given above, independently of one another.

### Biological/Pharmacological Properties

Through normalization of ghrelin receptor-dependent signaling within the striatal circuitry relevant for impulse control, GOAT inhibitors (such as compounds of general Formula I, including examples 1 to 167) may be effective in treating substance use disorders (SUD) in patients with high impulsivity. Individuals with opioid dependence often exhibit significant impairments in inhibitory control reflected in highly impulsive action, either because of chronic substance use or as a pre-existing trait that increases vulnerability to addiction (Verdejo-García, A., Lawrence, A. J., & Clark, L. Impulsivity as a vulnerability marker for substance-use disorders: review of findings from high-risk research, problem gamblers and genetic association studies. Neurosci. Biobehav. Rev.; 2008; 32(4), 777-810). Chronic opiate use leads to a preference for immediate rewards and impairs the ability to delay gratification (Tolomeo, S., Gray, S., Matthews, K., Steele, J. D., & Baldacchino, A. (2016). Multifaceted impairments in impulsivity and brain structural abnormalities in opioid dependence and abstinence. Psychol. Med; 2016; 46(13), 2841-2853). Even during sustained remission, patients struggle with impulsive actions (Psederska, E., & Vassileva, J. Neurocognitive Impulsivity in Opiate Users at Different Lengths of Abstinence. Int J Environ Res Public Health; 2023; 20(2), 1236.), significantly contributing to relapse (Jupp B, Dailey J W, Behavioral endophenotypes of drug addiction: etiological insights from neuroimaging studies. Neuropharmacology 2014; 76; 487-497). This underscores the need for targeted interventions, especially during early remission. Surprisingly, the novel potential of GOAT inhibitors to improve inhibitory control in SUDs is demonstrated by example 70, which enhances inhibitory control in high-impulsive rodents in the 5-choice serial reaction time task (5-CSRTT).

### Assessment of Efficacy on Impulsive Behavior Tested in the Rat Five Choice Serial Reaction Time Task (5-CSRTT)

Assessment of efficacy on motor impulsive behavior may be investigated as follows:

5-CSRTT task training took place according to standard protocols (Isherwood et al. Neuropharmacology 2017, 123: 249-260). Briefly, rats were trained to nose poke at the location of a light cue presented at 1 of 5 locations on a curved wall of an operant box (Med Associates Inc, St. Albans, Vermont). If a nose poke occurred at the illuminated location during or up to 1 s after stimulus presentation a sugar pellet was delivered in a reward receptacle located across the chamber. Infrared beams in each choice aperture and the reward receptacle allowed for precise detection of the rat at this task associated operanda. Motor impulsive behavior was defined as a response at any nose poke aperture which occurred before onset of the light cue (premature response).

After reaching stable performance, a new analytical approach was applied which revealed trait-like (long-term) stability in the number of premature responses individual animals made across several months. In general, this analysis made it possible to robustly stratify animals into high- and low-impulsive groups based on longitudinal assessment of the number of premature responses they made during training.

Experiments were performed in cross-over such that all experiment subjects received both vehicle and compound, on separate days, with each administration separated by ˜2 weeks. The order of vehicle and compound administration was randomized within experimental subjects, while a third group was administered Atomoxetine on both experimental days as a technical control.

As a standardized numerical threshold for impulsivity levels, animals with >40 and <40 premature responses (out of 200 initiated trials) in vehicle were labeled as high and low impulsive, respectively. Importantly, this numerical threshold-based labeling overlapped >80% with the longitudinal analysis of the training data (described above). The high convergence of these two approaches towards stratification allowed us to robustly compare compound effects in stably high- vs stably low-impulsive rats in the 5-CSRTT.

TABLE 1
Test results (example 70 was used as the test compound “cpd”)

| | |
|---|---|
| Impulsivity | Impulsivity (Premature Counts) |

| level of | | cpd 0.15 | cpd 0.5 | cpd 1.5 |
|---|---|---|---|---|
| animal group | Vehicle | mg/kg | mg/kg | mg/kg |
| High | 32 ± 3 | 19 ± 3 | 19 ± 2 | 16 ± 3 |
| Low | 11 ± 1 | 15 ± 2 | 17 ± 2 | 16 ± 2 |

The above data of table 1 shows that the compound of example 70 significantly reduces the impulsivity in high impulsive rats at all three tested doses.

The psychological and neural basis of impulsivity can be studied and experimentally translated from humans to rodents. Striking parallels can be observed in the underlying neurobehavioral systems, which enable both macro- and micro-definition of functional circuits (Dailey J W, Robbins T W. Fractionating impulsivity: neuropsychiatric implications. Nat Rev Neurosci 2017; 18: 158-171).

In vivo, the compound of example 70 significantly reduced acylated ghrelin in plasma in rats. Treatment of rats with example 70 significantly reduced impulsive behavior in high impulsive animals in a translatable behavioral task (5-choice serial reaction time task) without observable confounding factors.

Early Real World Evidence suggests that improving impulsivity may translate to an improvement in addiction outcomes.

### Methods and Scales to Measure Impulsivity in Humans

To assess impulsivity, there are several objective measures that have a long history in experimental neuroscience and strong data to support the linkage of task behavior with specific corticostriatal brain circuits (Dailey J W, Robbins T W. Fractionating impulsivity: neuropsychiatric implications. Nat Rev Neurosci 2017; 18: 158-171). An objective measure of impulsivity (DDT) is proposed to assess the effect of example 70 in humans with additional exploratory measures of efficacy (e.g., craving, BIS-11, S—UPPS—P, the 4-CSRTT, cue reactivity test) also collected after 8 days of treatment. Considering the increased risk of negative addiction outcomes posed by heightened impulsivity as well as emerging Real World Evidence, it is considered that a significant reduction of impulsivity may suffice to demonstrate a meaningful improvement over available therapies.

### 4-Choice Serial Reaction Time Test (4-CSRTT)

The 4-CSRTT task is a computerized task of waiting impulsivity that was developed based on the rodent 5-CSRTT (Voon V, Irvine M A, Derbyshire K, et al. Measuring “waiting” impulsivity in substance addictions and binge eating disorder in a novel analogue of rodent serial reaction time task. Biol Psychiatry 2014; 75:148-155). Participants are required to press and hold down the space bar with their dominant index finger while four boxes are presented on the screen. After a specified period (cue-target interval), a green circle target appears briefly and randomly in 1 of the 4 boxes. Participants are instructed to release the space bar and touch the box in which the target appeared. Baseline blocks without monetary reward feedback are used to individualize subsequent blocks based on the individual's mean reaction time (RT) and standard deviation (SD). The subsequent 4 test blocks with monetary feedback are optimized to increase premature responding. This includes variation of target duration, variability of the cue-target interval, and the presence of distractors. Responses are followed by outcome feedback depending on performance. The task duration is 10-15 mins. The main outcome measure, premature responding, is defined as a release of the space bar before target onset. The short version consists of 2 trials blocks and a practice run. The standard version consists of 1 baseline block and 4 test blocks.

### Delay Discounting Task (DDT)

In the DDT, participants make choices on the screen between a smaller, immediate amount of money and a larger, delayed amount. Steep discounting of delayed rewards is indicative of cognitive impulsivity. A behavioral version of the task incudes that the amount of the delayed option is reduced in a stepwise manner after 2 successive choices of the delayed reward and increased in a stepwise manner after 2 successive choices of the immediate reward. The key outcome variable for this task is the discount rate (k) or rate at which delayed rewards lose their value. The DDT task duration is 10-15 mins. An MRI compatible version of the DDT can be completed in the scanner. By analyzing the neural correlates of decision-making in this task, researchers can gain insights into the brain processes underlying impulsive behavior and self-control.

### Barratt Impulsiveness Scale Version 11 (BIS-11)

The Barratt Impulsiveness Scale version 11 (BIS-11) is described in Vasconcelos, A. G., Malloy-Diniz, L., & Correa, H. (2012). Systematic review of psychometric proprieties of Barratt Impulsiveness Scale Version 11 (BIS-11). Clinical Neuropsychiatry: Journal of Treatment Evaluation, 9(2), 61-74.

### Short Urgency, Premeditation, Perseverance, Sensation Seeking, Positive Urgency Impulsive Behavior Scale (S—UPPS-P)

The Short Urgency, Premeditation, Perseverance, Sensation Seeking, Positive Urgency Impulsive Behavior Scale (S—UPPS-P) is described in Whiteside, S. P. and Lynam, D. R., The Five Factor Model and Impulsivity: Using a Structural Model of Personality to Understand Impulsivity. Personality and Individual Differences 2001, 30(4), 669-689.

### Opioid Craving Visual Analog Scale (OC-VAS)

The Opioid Craving Visual Analog Scale (OC-VAS) is described in Boynett et al., Drug Alcohol Depend (2021). Assessment of craving in opioid use disorder: Psychometric evaluation and predictive validity of the opioid craving VAS (DOI: 10.1016/j.drugalcdep.2021.109057.

### Cue Reactivity Test

An increased neural response to drug-related cues has been well documented in substance use disorder patients and is associated with drug craving, drug seeking, and relapse (Ekhtiari H, Kuplicki R, Aupperle R L, et al. It is never as good the second time around: brain areas involved in salience processing habituate during repeated drug cue exposure in treatment engaged abstinent methamphetamine and opioid users. Neuroimage; 2021; 238; 118180). In this task, participants will be presented with 4 blocks of drug-related pictures and 4 blocks of neutral pictures while undergoing fMRI scanning. After every block, participants are asked to rate their current urge to use drug on a one to four scale, with 1 being “No Urge” and 4 being “Strong Urge” using a 4 key response box. Opioid-related pictures are selected from the methamphetamine and opioid cue database provided by the Laureate Institute for Brain Research (Tulsa, OK, USA).

### Pharmaceutical Compositions

The dose range of the compounds of general formula I applicable per day is usually from 0.001 to 10 mg per kg body weight, for example from 0.01 to 8 mg per kg body weight of the patient. Each dosage unit may conveniently contain from 0.1 to 1000 mg, for example 0.5 to 500 mg.

The preferred dose range of compounds of general formula I including example 70 applicable per day is from 0.5 to 20 mg per day and patient.

The actual therapeutically effective amount or therapeutic dosage will of course depend on factors known by those skilled in the art such as age and weight of the patient, route of administration and severity of disease. In any case the compound or composition will be administered at dosages and in a manner which allows a therapeutically effective amount to be delivered based upon patient's unique condition.

The compounds, compositions, including any combinations with one or more additional therapeutic agents, according to the invention may be administered by oral, transdermal, inhalative, parenteral or sublingual route. Of the possible methods of administration, oral or intravenous administration is preferred. Most preferred is oral administration.

Suitable preparations for administering the compounds of formula I including example 70, optionally in combination with one or more further therapeutic agents, will be apparent to those with ordinary skill in the art and include for example tablets, pills, capsules, suppositories, lozenges, troches, solutions, syrups, elixirs, sachets, injectables, inhalatives and powders etc. Oral formulations, particularly solid forms such as e.g. tablets or capsules are preferred. The content of the pharmaceutically active compound(s) is advantageously in the range from 0.1 to 90 wt.-%, for example from 1 to 70 wt.-% of the composition as a whole.

Suitable tablets may be obtained, for example, by mixing one or more compounds according to formula I with known excipients, for example inert diluents, carriers, disintegrants, adjuvants, surfactants, binders and/or lubricants. The tablets may also consist of several layers. The particular excipients, carriers and/or diluents that are suitable for the desired preparations will be familiar to the skilled man on the basis of his specialist knowledge. The preferred ones are those that are suitable for the particular formulation and method of administration that are desired. The preparations or formulations according to the invention may be prepared using methods known per se that are familiar to the skilled man, such as for example by mixing or combining at least one compound of formula I according to the invention, or a pharmaceutically acceptable salt of such a compound, and one or more excipients, carriers and/or diluents.

### Combination Therapy

A compound of the invention may be administered as part of a combination therapy together with another active agent for the treatment of the disease or disorder in question, e.g. buprenorphine, methadone, L-methadone, naltrexone and/or naloxone substitution therapy.

In such cases, the two active agents may be given together or separately, e.g. as constituents in the same pharmaceutical composition or formulation, or as separate formulations.

The dosage for the combination partners mentioned above is usually ⅕ of the lowest dose normally recommended up to 1/1 of the normally recommended dose.

Therefore, in another aspect, this invention relates to the use of a compound according to the invention in combination with one or more additional therapeutic agents described hereinbefore and hereinafter for the treatment of diseases or conditions as described hereinbefore and hereinafter.

In yet another aspect the present invention relates a method for treating a disease or condition as described herein in a patient that includes the step of administering to the patient, preferably a human, in need of such treatment a therapeutically effective amount of a compound of the present invention in combination with a therapeutically effective amount of one or more additional therapeutic agents described in hereinbefore and hereinafter.

The use of the compound according to the invention in combination with the additional therapeutic agent may take place simultaneously or at staggered times.

The compound according to the invention and the one or more additional therapeutic agents may both be present together in one formulation, for example a tablet or capsule, or separately in two identical or different formulations, for example as a so-called kit-of-parts.

Consequently, in another aspect, this invention relates to a pharmaceutical composition which comprises a compound according to the invention and one or more additional therapeutic agents described hereinbefore and hereinafter, optionally together with one or more inert carriers and/or diluents.

Other features and advantages of the present invention will become apparent from the following more detailed Examples which illustrate, by way of example, the principles of the invention.

### ExampleS

### Example I

A Phase Ib trial is conducted to evaluate the effects on impulsivity, pharmacokinetics, safety, and tolerability of compound 70, given orally, in patients with opioid use disorder taking background buprenorphine treatment.

The original purpose of the trial was to examine preliminary efficacy of oral compound 70 in treating impulsivity by comparing the mean change from baseline in both the k parameter of the Delay Discounting Task (DDT) and the total number of premature responses in the 4 Choice Serial Reaction Time Test (4-CSRTT) at Day 8. The amended purpose of the trial is to examine preliminary efficacy of oral compound 70 in treating impulsivity by comparing the mean change from baseline in the k parameter of the Delay Discounting Task (DDT) at Day 8. The primary treatment comparison will be on treatment, which will assume all participants took randomized treatment.

Participants are put in 2 groups, one receiving the compound of example 70 (also called “example 70” or “compound 70”), the other receiving placebo.

After a screening period, participant are randomized.

### Trial Design

The trial is a randomized, double-blind (participant and investigator), placebo-controlled, parallel group trial to examine the preliminary efficacy and safety of oral compound 70 once daily over an 8-day treatment period in adult male participants with moderate to severe OUD and impulsivity that are on stable background buprenorphine-based medication for OUD (MOUD) treatment.

### Trial Medication

Compound 70 in an amount of 2.5 mg and matching placebo are supplied as film-coated tablets. Four of these tablets are admininstered once daily, resulting in a treatment dose of 10 mg qd.

Standard pharmaceutical excipients are selected for the manufacture of example 70 film-coated tablets which are commonly used in oral dosage forms.

The tablet cores of the formulation include lactose monohydrate (filler), microcrystalline cellulose (filler), hydroxypropylcellulose (binder), crospovidone (disintegrant), and magnesium stearate (lubricant); purified water is used as a processing aid (solvent) for the manufacture of the tablet cores and for film-coating of the cores (film-coating solvent). The film-coating mixture consists of hypromellose, macrogol, talc, titanium dioxide, and iron oxide red.

### Trial Population

In total, about 90 participants currently engaged in medications for opioid use disorder (MOUD) treatment based on buprenoprphine/naloxone administration are randomized into the trial.

Main Inclusion Criteria/Diagnosis:
Male participants, 18 to 65 years of age
Meet current DSM-5 diagnostic criteria for OUD, of at least moderate severity within the 12 months prior to screening
Currently engaged in medications for opioid use disorder (MOUD) treatment at a buprenorphine/naloxone sublingual film total daily dose ranging from 8 mg/2 mg to 24 mg/6 mg or buprenorphine/naloxone sublingual tablet from 5.7 mg/1.4 mg to 17.1 mg/4.3 mg daily, or buprenorphine sublingual tablet from 8 mg to 24 mg total daily dose for at least 2 weeks at screening OR on a stable dose of depot injectable buprenorphine for at least 5 weeks at screening, with at least 1 week since last depot buprenorphine injection
Have a current MOUD prescription and a positive urine drug screen to document buprenorphine use
Willingness to abstain from using alcohol for 24 h and all other drugs of abuse for 72 h prior to Day 1 and through discharge from the trial site on Day 9

As used herein “current” in connection with DSM-5 diagnostic criteria for OUD means as of September 2024.

Main Exclusion Criteria:
Lifetime diagnosis of schizophrenia, schizoaffective disorder, schizophreniform disorder, bipolar I disorder, delusional disorder, or autism spectrum disorder
Moderate or severe substance use disorder other than OUD within the 6 months prior to screening (excluding tobacco, caffeine, and moderate stimulant use)
Severe stimulant use disorder within the 6 months prior to screening
Any other psychiatric disorder that is not currently stable in symptoms and treatment. Stable is defined as having no significant changes in symptom acuity or treatment (medication or psychotherapy treatment) in the 6 weeks prior to randomization
Currently experiencing a moderate to severe major depressive episode
Positive results on a urine drug screen for 3 or more drugs at screening (not counting buprenorphine)
Any positive result on a urine drug screen upon admission to the trial site (excluding buprenorphine and cannabis)
Intoxication at screening or randomization
Participants deemed to be at significant risk of serious violence or suicide
Concomitant use of psychotropic medication except for:
   Buprenorphine-based MOUD
   A single SSRI or SNRI antidepressant that has been stable in agent, dose, and frequency for more than 6 weeks prior to randomization
   Quetiapine (at a daily dose not exceeding 200 mg) and/or gabapentin, provided they have been stable in dose and frequency for more than 6 weeks prior to randomization
   A single sleep medication given as a nightly scheduled medication that has been stable in agent, dose, and frequency for more than 6 weeks prior to randomization. Allowed sleep medications include: non-benzodiazepine sleep medications (i.e. “Z-drugs”), antihistamines, melatonin, trazodone, or doxepin
Current Type 1 or Type 2 diabetes mellitus
History of seizure disorders, stroke, brain tumor, or any other major neurological illness
Any contraindication to undergo magnetic resonance imaging (MRI) radiography (e.g. history of surgery involving metal implants) or that could interfere with the performance of behavioral or functional MRI tasks, including the following:
   Participants who have undergone operations to the head
   Participants with significant hearing impairments
   Participants with a significant visual impairment, or history of ocular treatment including corrective laser eye surgery
   Participants with a weight or body circumference that exceeds MRI scanner capabilities or a BMI of ≥35 kg/m²
A positive result for an active hepatitis B or C infection

### Trial Endpoints

Primary endpoint:
Change from baseline in k parameter of the DDT

There are no secondary endpoints for this trial.

Further endpoints:

### Efficacy

Change from baseline in total number of premature responses in the 4-CSRTT at Day 8
Change from baseline in the Opioid Craving Visual Analog Scale (OC-VAS)
Change from baseline in the Barratt Impulsiveness Scale (BIS-11) total score
Change from baseline in the Short Urgency, Premeditation, Perseverance, Sensation Seeking, Positive Urgency Impulsive Behavior Scale (S—UPPS-P) total score

### Biomarkers

Change from baseline of acylated ghrelin (AG), unacylated ghrelin (UAG), and the AG/UAG ratio in plasma following administration of compound 70.

### Statistical Methods

ANCOVA will be used for the analysis of the primary endpoint. These models will include fixed effects for treatment, and baseline value of the relevant endpoint.

1. A method for treating a patient with substance use disorder, the method comprising administering to the patient a pharmaceutically effective amount of a compound of formula (I) 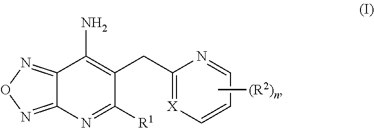
wherein
X is CH or N;
R¹ is selected from the group consisting of CH₃, —CH₂OH and Cl;
R² is independently of each other selected from the group consisting of H, F, Cl, Br, I, CN, C₁₋₆-alkyl, C₃₋₇-cycloalkyl, OH, —O—(C₁₋₆-alkyl), —O—(C₃₋₇-cycloalkyl), —O—(C₁₋₃-alkyl)-(C₃₋₇-cycloalkyl), —O-heterocyclyl, —O—(C₁₋₃-alkyl)-heterocyclyl, —O-aryl, —O-heteroaryl, —S—(C₁₋₃-alkyl), —SO—(C₁₋₃-alkyl), —SO₂-(C₁₋₃-alkyl), —NH₂, —NH—(C₁₋₆-alkyl), —NH—(C₃₋₆-cycloalkyl), —NH—(C₁₋₃-alkyl)-heterocyclyl, —NH—(C₁₋₆-alkyl)-C(═O)—NH₂, —C(═O)—NH₂, —C(═O)—NH—(C₁₋₃-alkyl), —C(═O)—N(C₁₋₃-alkyl)₂, —C(═O)OH, —C(═O)—O—(C₁₋₄-alkyl), —C(═O)-(C₁₋₄-alkyl), —C₁₋₃-alkyl-C(═O)—O—(C₁₋₄-alkyl), heterocyclyl, heteroaryl and 5,6-dihydro-4H-pyrrolo[1,2-b]pyrazol-3-yl,
wherein each alkyl or cycloalkyl group is optionally independently substituted with one or more substituents selected from the group consisting of F, CN and OH, and
wherein each heterocyclyl group is selected from a mono- or spirocyclic 4-7-membered cycloalkyl group, in which 1, 2 or 3 CH₂-groups are independently of each other replaced by O, S, NH or C═O, and
wherein each heterocyclyl group is optionally substituted with 1 or 2 substituents independently of each other selected from F, OH and C₁₋₃-alkyl,
wherein each aryl group is selected from a group consisting of phenyl and naphthyl, and
wherein each heteroaryl group is selected from a 5-membered aromatic cycle containing 1 or 2 heteroatoms independently selected from N, O and S or from a 6-membered aromatic cycle containing 1 or 2 N, and
wherein each aryl or heteroaryl group is optionally substituted with 1 or 2 substituents independently selected from a group consisting of F, CN and C₁₋₃-alkyl, which is optionally substituted with one or more F;
or, if two groups R² are attached to adjacent C atoms of the pyridine or pyrimidine group, they may be linked with each other and together form a —O—CH₂—O—, —O—CH₂—CH₂—O— or —O—CH₂—CH₂—CH₂—O— bridge, in which 1 or 2 H atoms may be replaced with F or C₁₋₃-alkyl; and
n is 1, 2 or 3;
wherein each of the above-mentioned alkyl groups may be substituted with one or more F;
or a pharmaceutically acceptable salt thereof.

2. The method according to claim 1, wherein
R¹ is —CH₃; and
n is 1 or 2,
or a pharmaceutically acceptable salt thereof.

3. The method according to claim 1, wherein
R² is independently of each other selected from the group consisting of H, F, Cl, Br, CN, C₁₋₆-alkyl, C₃₋₇-cycloalkyl, OH, —O—(C₁₋₆-alkyl), —O—(C₁₋₃-alkyl)-(C₃₋₇-cycloalkyl), —O— heterocyclyl, —O—(C₁₋₃-alkyl)-heterocyclyl, —O-aryl, —O-heteroaryl, —S—(C₁₋₃-alkyl), —SO₂-(C₁₋₃-alkyl), —NH₂, —NH—(C₁₋₆-alkyl), —NH—(C₃₋₆-cycloalkyl), —NH—(C₁₋₃-alkyl)-heterocyclyl, —NH—(C₁₋₆-alkyl)-C(═O)—NH₂, —C(═O)—NH₂, —C(═O)—NH—(C₁₋₃-alkyl), —C(═O)-(C₁₋₄-alkyl), —C₁₋₃-alkyl-C(═O)—O—(C₁₋₄-alkyl), heterocyclyl, heteroaryl and 5,6-dihydro-4H-pyrrolo[1,2-b]pyrazol-3-yl,
wherein each alkyl or cycloalkyl group is optionally independently substituted with one or more substituents selected from the group consisting of F, CN and OH, and
wherein each heterocyclyl group is selected from a mono- or spirocyclic 4-7-membered cycloalkyl group, in which 1, 2 or 3 CH₂-groups are independently of each other replaced by O, S, NH or C═O, and
wherein each heterocyclyl group is optionally substituted with 1 or 2 substituents independently of each other selected from F, OH and C₁₋₃-alkyl,
wherein each aryl group is selected from a group consisting of phenyl and naphthyl, and
wherein each heteroaryl group is selected from a 5-membered aromatic cycle containing 1 or 2 heteroatoms independently selected from N, O and S or from a 6-membered aromatic cycle containing 1 or 2 N, and
wherein each aryl or heteroaryl group is optionally substituted with 1 or 2 substituents independently selected from a group consisting of F and C₁₋₃-alkyl, which is optionally substituted with one or more F;
or, if two groups R² are attached to adjacent C atoms of the pyridine or pyrimidine group, they may be linked with each other and together form a —O—CH₂—O—, —O—CH₂—CH₂—O— or —O—CH₂—CH₂—CH₂—O— bridge,
or a pharmaceutically acceptable salt thereof.

4. The method according to claim 1, wherein
R² is independently of each other selected from the group consisting of F, Cl, Br, CN, C₁₋₃-alkyl, C₃₋₆-cycloalkyl, —O—(C₁₋₄-alkyl), —O—CH₂-cyclopropyl, —O—CH₂-heterocyclyl, —O— phenyl, —O-heteroaryl,-S—CH₃, —NH₂, —NH—(C₁₋₄-alkyl), —NH—(C₃₋₅-cycloalkyl), —NH—(CH₂-heterocyclyl), —NH—(C₁₋₄-alkyl)-C(═O)—NH₂, —C(═O)—NH—(C₁₋₃-alkyl), —C(═O)-(C₁₋₄-alkyl), heterocyclyl, heteroaryl and 5,6-dihydro-4H-pyrrolo[1,2-b]pyrazol-3-yl,
wherein each alkyl or cycloalkyl group is optionally independently substituted with one to three F atoms or with one CN or one OH, and
wherein each heterocyclyl group is selected from a group consisting of oxetanyl, tetrahydrofuranyl, azetidinyl, pyrrolidinyl, morpholinyl and 1,4-diazepan-5-one, and
wherein each heterocyclyl group is optionally substituted with 1 or 2 substituents independently of each other selected from F, OH and CH₃,
wherein each heteroaryl group is selected from a group consisting of furanyl, isoxazolyl, thiazolyl and pyrazolyl, and
wherein each heteroaryl group is optionally substituted with 1 or 2 substituents independently selected from a group consisting of F, CH₃ and CF₃,
or a pharmaceutically acceptable salt thereof.

5. The method according to claim 1, wherein
R² is independently of each other selected from the group consisting of F, Cl, Br, CN, CH₃, C₃₋₅-cycloalkyl, —O—(C₁₋₄-alkyl), —O—CH₂-heterocyclyl, —O-phenyl,-S—CH₃, —NH₂, —NH—(C₁₋₄-alkyl), —NH—(C₃₋₅-cycloalkyl), —NH—(CH₂-heterocyclyl), —NH—(C₁₋₄-alkyl)-C(═O)—NH₂, heterocyclyl, heteroaryl and 5,6-dihydro-4H-pyrrolo[1,2-b]pyrazol-3-yl,
wherein each alkyl or cycloalkyl group is optionally independently substituted with one to three F atoms or with one CN or one OH, and
wherein each heterocyclyl group is selected from a group consisting of oxetanyl, azetidinyl, pyrrolidinyl, morpholinyl and 1,4-diazepan-5-one, and
wherein each heterocyclyl group is optionally substituted with 1 or 2 substituents independently selected from F, OH and CH₃, and
wherein each heteroaryl group is selected from a group consisting of furanyl and thiazolyl,
or a pharmaceutically acceptable salt thereof.

6. The method according to claim 1, wherein
R² is independently selected from the group consisting of: 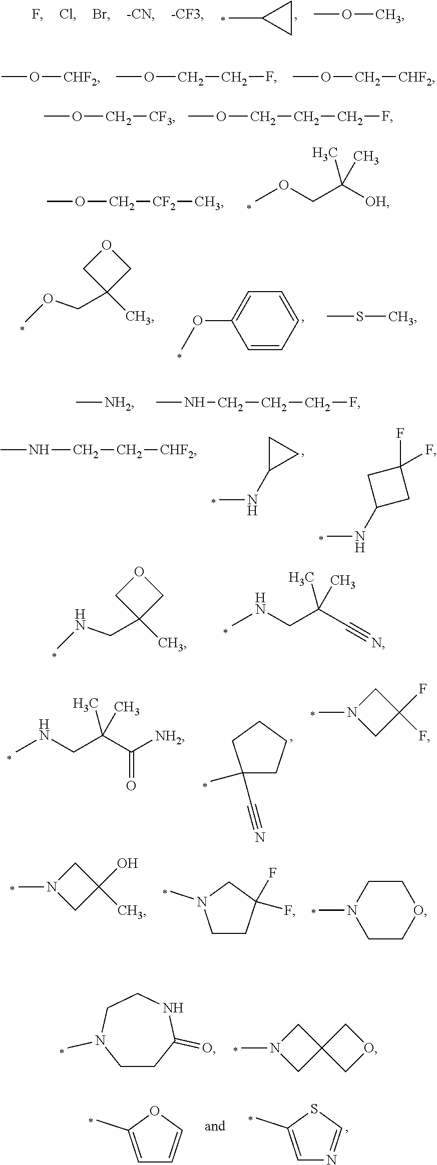
or a pharmaceutically acceptable salt thereof.

7. The method according to claim 1, wherein X is OH,
or a pharmaceutically acceptable salt thereof.

8. The method according to claim 1, wherein X is N,
or a pharmaceutically acceptable salt thereof.

9. The method according to claim 1 wherein compound (I) has the formula 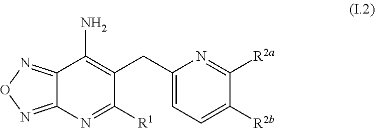
wherein
R¹ is CH₃;
R^{2a} and R^{2b} are each independently selected from the group consisting of: 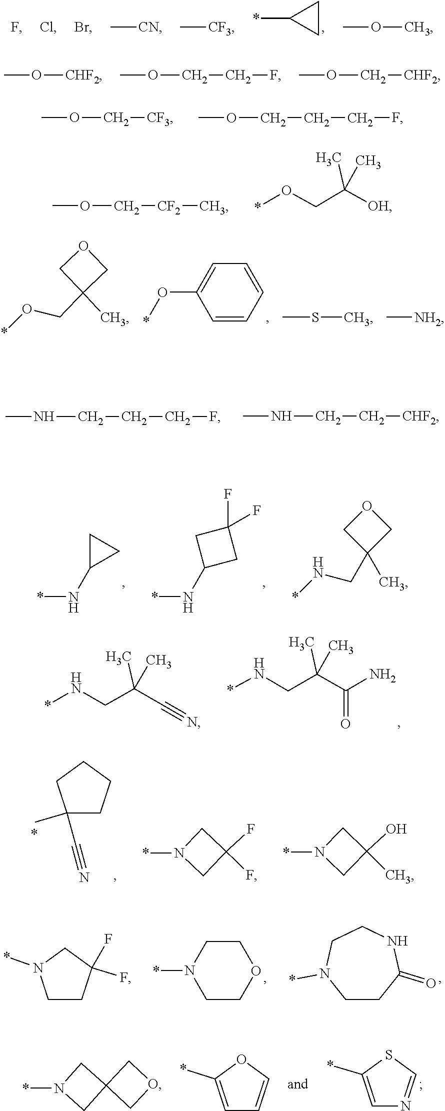
or a pharmaceutically acceptable salt thereof.

10. The method according to claim 1, wherein compound (I) is selected from the group consisting of: 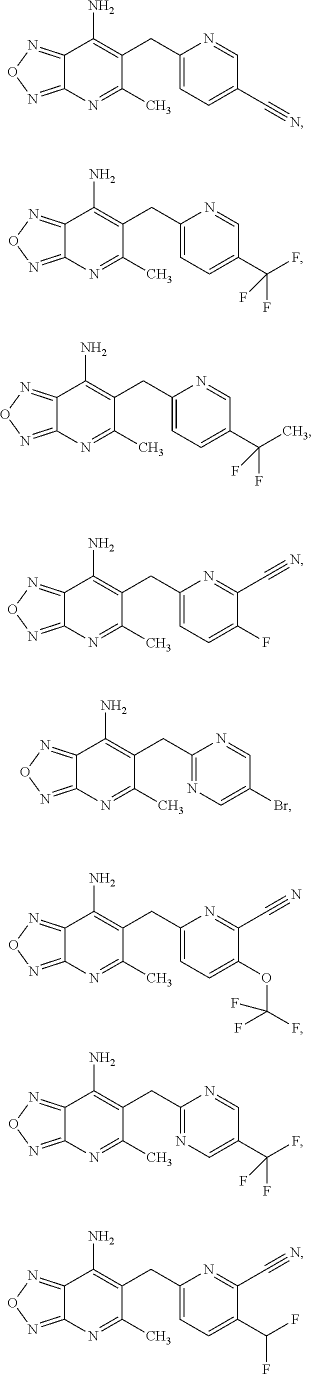 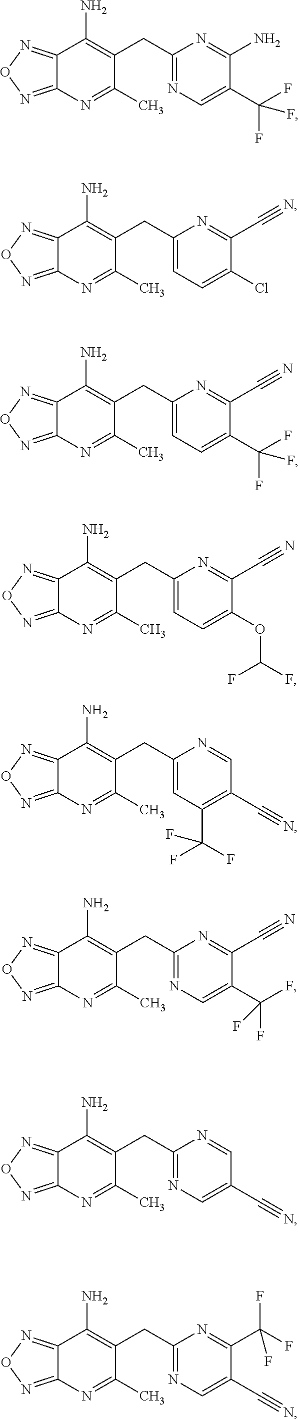 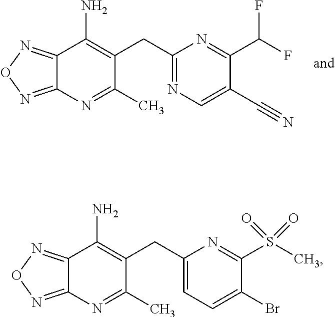 [SMILES: Cc1nc2nonc2c(N)c1Cc1ncc(C#N)c(C(F)F)n1 ; Cc1nc2nonc2c(N)c1Cc1ccc(Br)c(S(C)(=O)=O)n1]
or a pharmaceutically acceptable salt thereof.

11. The method according to claim 1, wherein the treatment is for:
Improving impulse control,
Normalizing impulsivity in substance use disorder patients with high impulsivity,
Improving the results of impulsivity tasks or scales selected from the group consisting of Delay Discounting Task (DDT), 4-Choice Serial Reaction Time Test (4-CSRTT), Barratt Impulsiveness Scale, version 11 (BIS-11), Short Urgency, Premeditation, Perseverance, Sensation Seeking, Positive Urgency Impulsive Behavior Scale (S—UPPS-P) and/or Opioid Craving Visual Analog Scale (OC-VAS),
Reducing or normalizing cue reactivity in substance use disorder patients with high impulsivity,
Reducing or normalizing addictive behaviors in substance use disorder patients with high impulsivity,
Reducing relapse after abstinence,
Reducing drug intake,
Improving quality of life,
Improving adherence and compliance with medications,
Improving adherence and compliance to psychotherapy,
Decreasing overall mortality and overdose mortality,
Reducing criminal behavior to satisfy drug consumption, and/or
Reducing craving, reducing drug use or abstaining from drug use or changing drug use pattern in substance use disorder (SUD), opioid use disorder (OUD) or stimulant use disorder (StimUD) patients with high impulsivity.

12. The method according to claim 1, wherein the substance use disorder is opioid use disorder.

13. The method according to claim 12, wherein the patient is taking buprenorphine as a background treatment.

14. The method according to claim 13, wherein the patient is an adult male.

15. The method according to claim 1, wherein the substance use disorder is stimulant use disorder.
